(19) United States
(12) Patent Application Publication
(10) Pub. No.: US 2026/0034070 A1
(43) Pub. Date: Feb. 5, 2026

(54) POLYMER NANOPARTICLE COMPOSITIONS FOR NON-VIRAL GENE DELIVERY

(71) Applicant: BATTELLE MEMORIAL INSTITUTE, Columbus, OH (US)

(72) Inventors: Caleb T. Hillrich, Columbus, OH (US); Kenneth R. Sims, JR., Columbus, OH (US); Emma K. Schmitz, Columbus, OH (US); Danielle J. Huk, Columbus, OH (US); Andrea D. McCue, Columbus, OH (US); Ashlee J. Colbert, Columbus, OH (US); Anthony D. Duong, Columbus, OH (US)

(21) Appl. No.: 19/291,060

(22) Filed: Aug. 5, 2025

Related U.S. Application Data
(60) Provisional application No. 63/679,415, filed on Aug. 5, 2024.
(60) Provisional application No. 63/693,367, filed on Sep. 11, 2024.
(60) Provisional application No. 63/775,094, filed on Mar. 20, 2025.

(51) Int. Cl. A61K 9/51; A61K 48/00; B82Y 5/00; C08F 293/00; C12N 15/88

(52) U.S. Cl. CPC A61K 9/5146; A61K 48/0041; C08F 293/00; C12N 15/88; B82Y 5/00; C08F 2438/03

(57) ABSTRACT

The disclosure relates to block copolymer nanoparticles for in vivo screening and for in vivo therapeutic delivery, and methods thereof. More particularly, the invention relates to polymer nanoparticles, such as reversible addition-fragmentation chain transfer (RAFT) polymer compositions, for delivering nucleotides.

### RELATED APPLICATIONS

This application claims the benefit of U.S. Provisional Application Nos. 63/775,094, filed Mar. 20, 2025, 63/693,367, filed Sep. 11, 2024, and 63/679,415, filed Aug. 5, 2024, the entire disclosure of each of which is incorporated herein by reference.

### BACKGROUND

Genetic medicines (including gene therapy, gene silencing, splicing regulators, and nuclease based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Adeno-associated viruses (AAVs) are the most widely used tool for genetic medicine delivery, but AAVs are not able to deliver large genetic payloads or multiple payloads (such as the clustered regularly interspaced short palindromic repeats (CRISPR)/Cas9 system), and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA), and a donor DNA strand at the same time to a particular location. This requirement becomes particularly limiting for in vivo applications because ensuring co-delivery of multiple large molecules to the same targeted location is currently not feasible. For example, the Cas9 enzyme sequence and guide RNA complex is too large to fit into AAVs.

Thus, there is a need for effective non-viral delivery systems, including gene delivery systems. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

### SUMMARY

In certain aspects, the disclosure is directed to block copolymers. In some embodiments, a block copolymer comprises
a first block having a monomer unit represented by formula I:

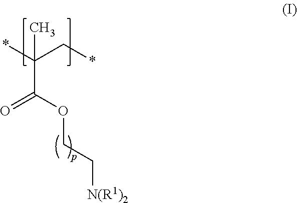

or a salt thereof, wherein:
   p is 0 or an integer selected from 1-3,
   each R¹ is individually selected from H or alkyl; and
   wherein each * represents a connecting point to the rest of the block copolymer; and
a second block comprising a copolymer of monomer units selected from formula II:

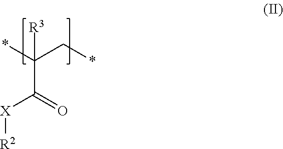

or a salt thereof, wherein:
   X is —O—;
   R² is alkyl; and
   R³ is alkyl;
   wherein each * represents a connecting point to the rest of the block copolymer; and
   formula III:

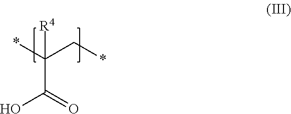

or a salt thereof, wherein:
   R⁴ is alkyl;
   wherein each * represents a connecting point to the rest of the block copolymer.

In some embodiments, the second block of the block copolymer has:
(i) a C Log P greater than about 1.5,
(ii) a Tg less than about 45° C.,
(iii) a hydrogen HSP less than about 5.4,
(iv) a surface energy of less than about 34,
(v) a surface energy of greater than about 38, or
(vi) a combination thereof with the exception of a combination of (iv) and (v).

In certain embodiments, a block copolymer comprises a first block and a second block, wherein:
the first block comprises a homopolymer of poly dimethylaminoethyl methacrylate p(DMAEMA), and
the second block comprises a copolymer of an alkylacrylate (e.g., butylmethacrylate (BMA)) and an acrylic acid (e.g., ethylacrylic acid (EAA) or propylacrylic acid (PAA));
wherein the first block is about 40% to about 80% by weight of the block copolymer.

In certain aspects, a polymer nanoparticle comprises:
a block copolymer according to the present disclosure wherein the nanoparticle has a diameter of about 15 nm to about 200 nm.

In certain aspects, a composition comprises:
a polymer nanoparticle according to the present disclosure, and
a nucleic acid complexed to the polymer nanoparticle.

In certain aspects, a method of treating a disease comprising administering a therapeutically effective amount of a composition according to the present disclosure to a patient in need thereof.

In certain aspects, a method of transfecting a cell comprising contacting the cell with an effective amount of a composition according to the present disclosure.

Additional embodiments, features, and advantages of the disclosure will be apparent from the following detailed description and through practice of the disclosure.

### BRIEF DESCRIPTION OF THE DRAWINGS

FIG. 1 shows encapsulation efficiency of the polymer nanoparticles complexed with mRNA expressing Cre recombinase, at a mass ratio of 30 to 1 (polymer to mRNA), plotted against the molar averaged C Log P of the block 2 of the polymer. All particles encapsulated a significant amount of the mRNA, with >80% of the mRNA mass excluded from the ribogreen dye.

FIG. 2 shows the log fold difference in PNP size upon loading with Cre mRNA, for the same PNPs as shown in FIG. 1, plotted against the molar averaged C Log P of the block 2 of the polymer. The two polymer families with the highest block 2 C Log P values, p(DMAEMA)-b-p(BMA-co-EAA) and p(DMAEMA)-b-p(BMA-co-PAA), were able to retain their small particle size upon mRNA loading, with a log fold difference of less than 0.

FIG. 3 shows transfection efficiency of the PNPs plotted against the log fold difference in PNP size upon loading with Cre mRNA. The PNPs that fell to the left of the zero line (i.e., the ones that less than doubled their size) also showed the highest transfection efficiencies, with 8 out of 9 of these PNPs having a % TE of greater than 10%. Conversely, the PNPs that fell to the right of the zero line (i.e. the ones that more than doubled their size after mRNA loading) also had the lowest % TE, with 17 out of 20 of these PNPs having a % TE of less than 10%.

FIG. 4 shows the change in PNP size upon loading with Cre mRNA, plotted against the molar averaged C Log P of Block 2 for the additional 64 PNPs described in Table 6. This figure includes PNPs 15-18 and 27-29 for comparison. All PNPs that had less than doubling in their size after mRNA loading had C Log P values greater than about 1.5.

FIG. 5 shows the change in PNP size upon loading with Cre mRNA, plotted against the Block 2 Molecular weight, for the additional 64 PNPs described in Table 6. This figure includes PNPs 15-18 and 27-29 for comparison. All of the PNPs that had less than doubling in their size after mRNA loading had a Block 2 molecular weight of greater than about 1.9 kDa.

FIG. 6. shows the change in PNP size upon loading with Cre mRNA for all 93 PNPs disclosed herein, plotted as a bubble plot, where the x-axis is the molar averaged C Log P of the block 2, the y-axis is the molecular weight of the block 2, and the size of the bubbles are proportional to the log fold change in PNP diameter after loading with Cre mRNA. All of the PNPs that had less than doubling in their size after mRNA loading had a C Log P value of greater than about 1.5 or a block 2 molecular weight of greater than about 1.2 kDa.

FIG. 7 shows effect of glass transition temperature on change in diameter of PNPs post loading.

FIG. 8 shows the effect of Hydrogen HSP on change in diameter of PNPs post loading.

FIG. 9 shows the effect of surface energy temperature on change in diameter of PNPs post loading

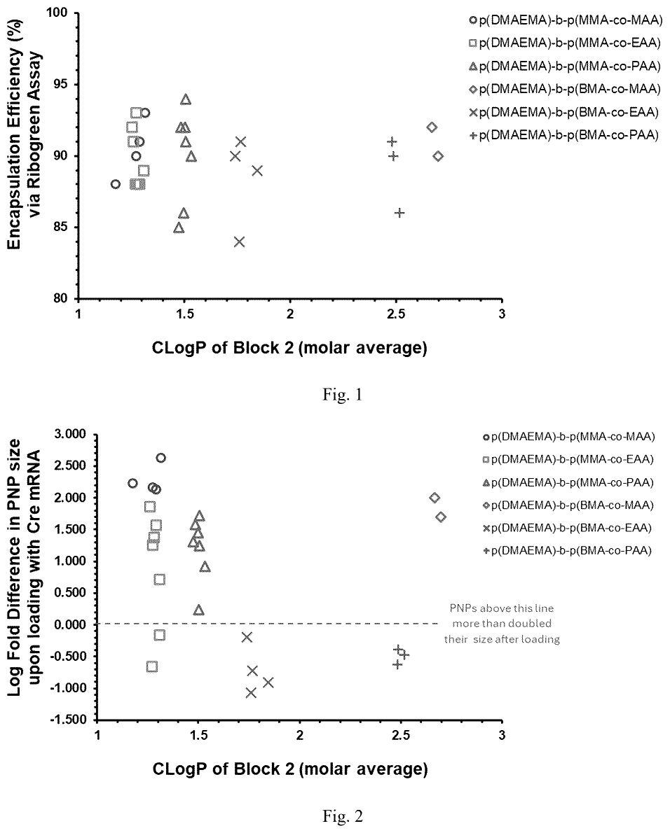

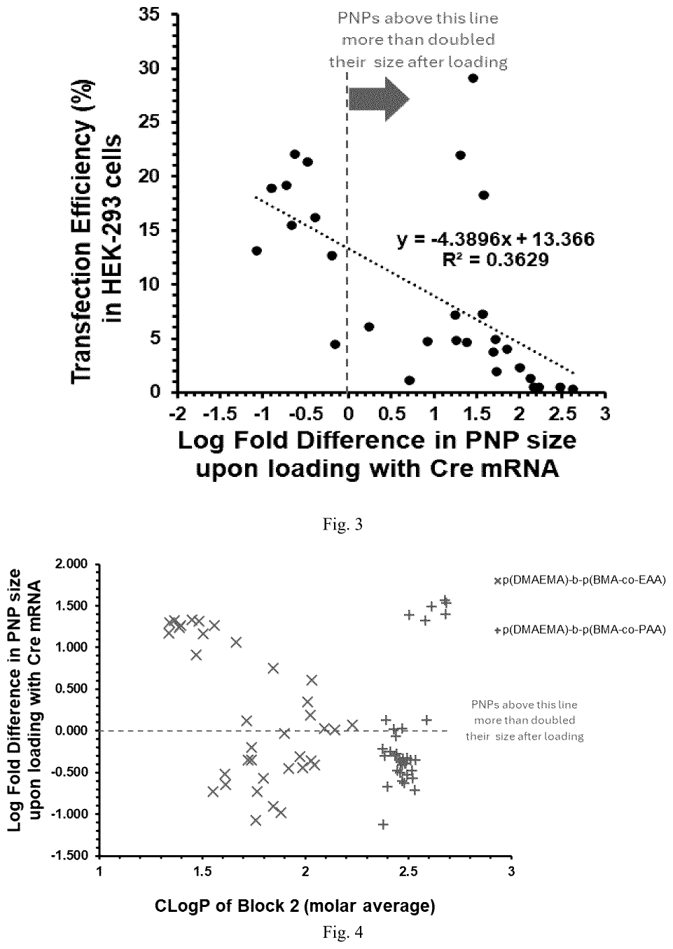

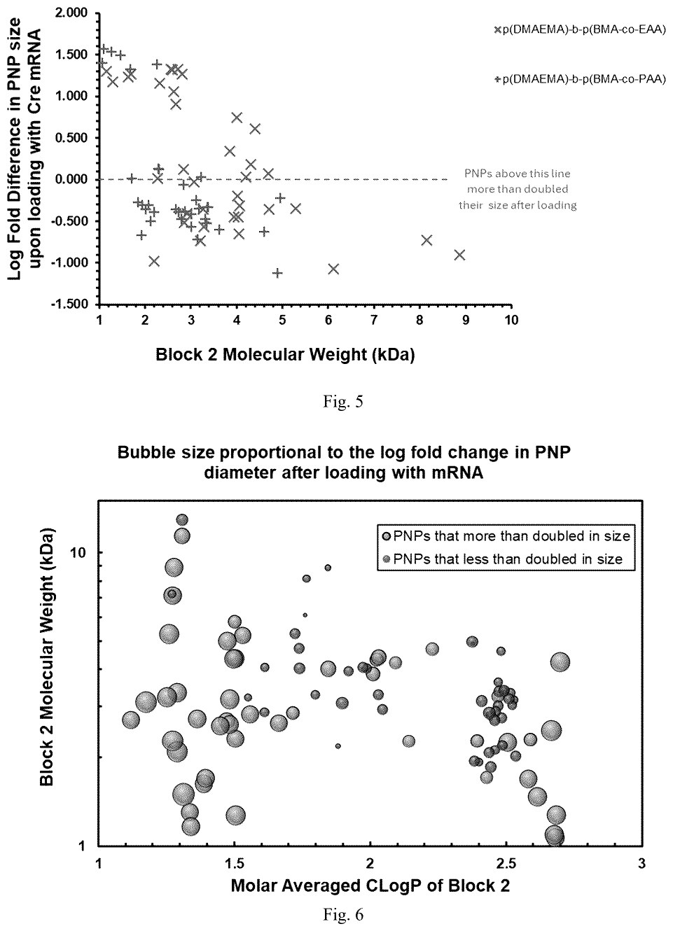

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” may be an approximation of ±10%, ±5%, or ±1%. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by“A-*”, “A-**”, or

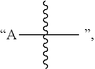

where each of “-*”, “-**”, and

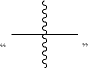

represents a bond to A and the point of covalent bond attachment to B. Alternatively, in some embodiments, the portion of A-B defined by the group or chemical structure B can be represented by “*-B”, “**-B”, or

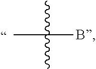

where each of “-*”, “-*” and

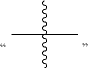

represents a bond to B and the point of covalent bond attachment to A.

As used herein, recitations of “molecular weight” regarding the first block refer to the weight average molecular weight (Mw) determined by a conventional polystyrene standard curve gel permeation chromatography method (hereinafter referred to as GPC) using multiple narrow distribution polystyrene standard samples relevant to the present invention and preferably between 2 kDa to 1,000 kDa. Recitations of “molecular weight” regarding the total molecular weight of the copolymer refer to a molecular weight from NMR spectra by calculating the relative amounts of each monomeric building block in the final diblock copolymer, as described in the Examples.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such subcombination of chemical groups was individually and explicitly disclosed herein.

### Chemical Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ-(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

The disclosure also includes acceptable salts (e.g., pharmaceutically acceptable salts) of the block copolymers described herein, preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. ⊖,” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

It will be understood that the chemical entities described herein, can exist as a salt of a free acid or base of a compound represented herein and an inorganic or organic counter ion.

Illustratively, the salt can be formed during the manufacture of the compound (e.g., a salt or a pharmaceutically acceptable salt) or can substituted to a salt for further manufacture, formulation, or administration reasons. As illustrated herein, certain compounds include a “W^{⊖},” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a block copolymer that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

### REPRESENTATIVE EMBODIMENTS

This disclosure describes compositions of cationic polymers (e.g., block copolymers, such as diblock copolymers) which self-assemble in aqueous conditions to form polymer nanoparticles (PNPs). In illustrative embodiments, the PNPs can complex nucleic acids and deliver the complexed nucleic acids to a desired location. In illustrative embodiments, the diameter of the PNPs is similar to the diameter of the PNPs when complexed to nucleic acids.

In certain embodiments, a block copolymer comprises a first block and a second block. The first block may comprise a homopolymer of poly dimethylaminoethyl methacrylate p(DMAEMA). The second block may comprise copolymer, such as a statistical copolymer (e.g., a random copolymer). For example, the second block may comprise a copolymer of an acrylate (e.g., methylmethacrylate or butylmethacrylate) and an acrylic acid (e.g., ethylacrylic acid (EAA) or propylacrylic acid (PAA)).

In certain embodiments, a block copolymer includes a first block having a monomer unit represented by formula I:

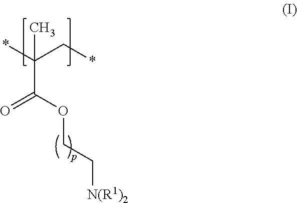

or a salt thereof, wherein:
   p is 0 or an integer selected from 1-3,
   each R¹ is individually selected from H or alkyl; and
wherein each * represents a connecting point to the rest of the block copolymer.

In certain embodiments, p is 0. In certain embodiments, p is 1, 2 or 3, and is preferably 1.

In certain embodiments, each R¹ is individually selected from H or alkyl (e.g., C₁-C₃ alkyl).

In certain embodiments, each R¹ is alkyl (e.g., C₁-C₃ alkyl) and each alkyl (e.g., C₁-C₃ alkyl) may be the same. In certain preferred embodiments, each R¹ is methyl. In certain embodiments, the first block comprises a plurality monomers of formula I such as

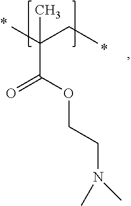

the corresponding polymer of which may be called poly(2-(Dimethylamino)ethyl methacrylate) p(DMAEMA).

In certain embodiments, a block copolymer comprises a copolymer second block comprising a copolymer. The second block may comprise a copolymer formed of two or more monomer units, each individually represented by formula II:

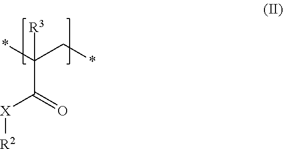

or a salt thereof, wherein:
   X is —O—;
   R² is alkyl; and
   R³ is alkyl;
   wherein each * represents a connecting point to the rest of the block copolymer; and
   formula III:

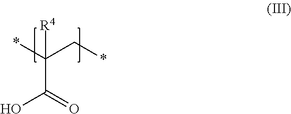

or a salt thereof, wherein:
   R⁴ is alkyl;
wherein each * represents a connecting point to the rest of the block copolymer. For example, the copolymer can be a statistical copolymer (e.g., a random copolymer).

In certain embodiments, R² is alkyl, such as C₁-C₆ alkyl (e.g., methyl or butyl), and is preferably butyl. For example, the monomer represented by formula II may be:

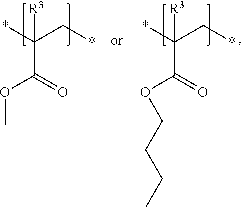

or a salt thereof.

In certain embodiments, R³ is alkyl such as C₁-C₆ alkyl, and is preferably methyl. For example, the monomer unit represented by formula II may be:

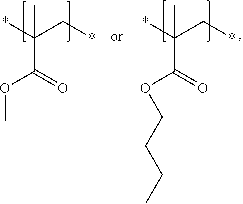

or a salt thereof, which may be referred to as methylmethacrylate (MMA) or butylmethacrylate (BMA), respectively, when present in the block copolymer.

In certain embodiments, R⁴ is alkyl such as C₁-C₆ alkyl (e.g., ethyl or propyl), and is preferably ethyl. For example, the monomer unit represented by formula III may be:

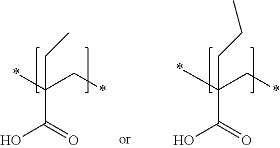

or a salt thereof, which may be referred to as ethylacrylic acid (EAA) or propylacrylic acid (PAA), respectively, when present in the block copolymer.

In certain embodiments, a block copolymer comprises a first block and a second block, the first block may comprise a homopolymer of poly dimethylaminoethyl methacrylate p(DMAEMA), and the second block may comprise a copolymer of an alkylacrylate (e.g., butylmethacrylate (BMA)) and an acrylic acid (e.g., ethylacrylic acid (EAA) or propylacrylic acid (PAA)), and the first block is about 40% to about 95%, about 40% to about 90%, about 50% to about 95%, about 50% to about 90%, about 50% to about 80%, about 60% to about 80%, or about 50% to about 70% by weight of the block copolymer.

In certain embodiments, a block copolymer comprises a first block and a second block, the first block may comprise a homopolymer of poly dimethylaminoethyl methacrylate p(DMAEMA), and the second block may comprise a copolymer of an alkylacrylate (e.g., butylmethacrylate (BMA)) and an acrylic acid (e.g., ethylacrylic acid (EAA) or propylacrylic acid (PAA)), and the first block is about 40% to about 80% or about 50% to about 70% by weight of the block copolymer.

In certain embodiments, a block copolymer comprises a first block and a second block, the first block may comprise a homopolymer of poly dimethylaminoethyl methacrylate p(DMAEMA), and the second block may comprise a copolymer of an alkylacrylate (e.g., butylmethacrylate (BMA)) and an acrylic acid (e.g., ethylacrylic acid (EAA) or propylacrylic acid (PAA)), and the first block is about 40% to about 90%, about 40% to about 80%, or about 50% to about 70% by weight of the block copolymer.

In certain embodiments, the first block has a molecular weight of about 4,000 Da to about 60,000 Da. In some embodiments, the first block has a molecular weight of about 4,000 Da to about 50,000. In some embodiments, the first block has a molecular weight of about 4,000 Da to about 40,000. In some embodiments, the first block has a molecular weight of about 4,000 Da to about 30,000. In some embodiments, the first block has a molecular weight of about 4,000 Da to about 20,000.

In certain embodiments, the first block has a molecular weight of about 4,000 Da to about 15,000 Da. For example, the first block may be about 4,000 Da to about 13,000 Da, or about 5,000 Da to about 10,000 Da.

In certain embodiments, the first block has a degree of polymerization of about 15 to about 400. In certain embodiments, the first block has a degree of polymerization of about 15 to about 350. In certain embodiments, the first block has a degree of polymerization of about 150 to about 400, about 150 to about 350, or about 200 to about 350. In certain embodiments, the first block has a degree of polymerization of about 30 to about 150. For example, the first block may have a degree of polymerization of about 30 to about 130, about 30 to about 110, about 30 to about 90, or about 30 to about 60.

In some embodiments, the second block has a molecular weight of about 1 kDa to about 18 kDa. In some embodiments, the second block has a molecular weight of about 1 kDa to about 13 kDa. In some embodiments, the second block has a molecular weight of about 2 kDa to about 5 kDa, for example about 2 kDa to about 4 kDa or about 3 kDa to about 5 kDa. In some embodiments, the second block has a molecular weight of about 5 kDa to about 18 kDa, for example about 5 kDa to about 15 kDa or about 5 kDa to about 10 kDa.

In certain embodiments, the second block has a molecular weight of about 1,000 Da to about 13,000 Da. In certain embodiments, the second block has a molecular weight of about 1,000 Da to about 12,000 Da. In some embodiments, the second block has a molecular weight of about 1,000 Da to about 10,000 Da. In some embodiments, the second block has a molecular weight of about 2,000 Da to about 7,000 Da.

In certain embodiments, the polymer includes a second block comprising a copolymer of BMA and EAA (ratio of about 25:75 to about 70:30 (e.g., molar ratio)), and has a second block molecular weight of greater than 2.2 about kDA. The second block molecular weight can be about 2.2 kDa to about 10 kDa. The log fold difference in PNP size upon loading with Cre mRNA of a polymer comprising such a second block can be less than 0.

In certain embodiments, the polymer includes a second block comprising copolymer of BMA and PAA (ratio of about 25:75 to about 70:30 (e.g., molar ratio)), and has a second block molecular weight of greater than 1.8 about kDA. The second block molecular weight can be about 1.8 kDa to about 10 kDa. The log fold difference in PNP size upon loading with Cre mRNA of a polymer comprising such a second block can be less than 0.

In certain embodiments, the molecular weights of the compound of formula II (e.g., BMA) and the compound of formula III (e.g., EAA or PAA) in the second block may be present at a particular ratio. For example, in certain embodiments the ratio of molecular weights of the monomers of formula II and the monomers of formula III in the second block is about 30:70 to about 90:10, about 40:60 to about 75:25, or about 50:50 to about 75:25. In certain embodiments, the ratio of molecular weights of the monomers of formula II and the monomers of formula III in the second block is about 60:40.

In certain embodiments, the monomer units of the compound of formula II (e.g., BMA) and the compound of formula III (e.g., EAA or PAA) in the second block may be present at a particular molar ratio. For example, in certain embodiments the molar ratio of the monomers of formula II and the monomers of formula III in the second block is about 30:70 to about 90:10, about 40:60 to about 75:25, or about 50:50 to about 75:25. In certain embodiments, the molar ratio of the monomers of formula II and the monomers of formula III in the second block is about 60:40.

In certain embodiments of the second block, the compound of formula II (e.g., MMA or BMA) and the compound of formula III (e.g., MAA, EAA or PAA) in the second block may be present at a particular molar ratio. For example, in certain embodiments the molar ratio of the monomers of formula II and the monomers of formula III in the second block is about 30:70 to about 90:10, about 40:60 to about 75:25, or about 50:50 to about 75:25. In certain embodiments, the molar ratio of the monomers of formula II and the monomers of formula III in the second block is about 60:40.

In certain embodiments, the second block has a degree of polymerization of about 15 to about 150. In certain embodiments, the second block has a degree of polymerization of about 15 to about 100. In certain embodiments, the second block has a degree of polymerization of about 15 to about 85. In certain embodiments, the second block has a degree of polymerization of about 15 to about 60. For example, the second block may have a degree of polymerization of about 15 to about 36, about 25 to about 35, about 35 to about 45, or about 45 to about 55. In certain embodiments, the second block has a degree of polymerization of about 15 to about 25.

In certain embodiments, the second block has (i) a C Log P greater than about 1.5, (ii) a Tg less than about 45° C., (iii) a hydrogen HSP less than about 5.4, (iv) a surface energy of less than about 34, (v) a surface energy of greater than about 38, or (vi) a combination thereof with the exception of a combination of (iv) and (v). For example, the second block can have at least two of (i)-(v), at least three of (i)-(v), or four of (i)-(v), in any combination thereof. In some embodiments, the second block has (i) and (ii), (i) and (iii), (i) and (iv), or (i) and (v). In some embodiments, the second block has (ii) and (iii), (ii) and (iv), or (ii) and (v). In some embodiments, the second block has (iii) and (iv) or (iii) and (v). In some embodiments, the second block has (i), (ii), and (iii); (i), (ii), and (iv); (i), (ii), and (v); (i), (iii), and (iv); (i), (iii), and (v); (ii), (iii), and (iv); or (ii), (iii), and (v). In some embodiments, the second block (i), (ii), (iii), and (iv); or (i), (ii), (iii), and (v). It should be understood that the values described herein for (i), (ii), (iii), (iv), and (v) may be applicable to each of the described combinations.

In certain embodiments, the second block has a C Log P of at least about 1.5, at least about 1.6, for example at least about 1.7. For example, the C Log P of the second block may be about 1.5 to about 3, about 1.5 to about 2.6, or about 1.6 to about 2.6. In some embodiments, the C Log P of the second block may be about 2 to about 3 or about 2 to about 2.5. To determine the C Log P of the block, the C Log P of each monomer was assigned the value of X Log P3 listed in the PubChem database. For each block of the polymer, the values were calculated according to the measured monomer ratios, as described herein.

In certain embodiments, the second block has a hydrogen Hansen Solubility Parameter (HSP) value of less than about 5.4. For example, the second block can have a hydrogen HSP value of about 3 to about 5.4 or about 4 to about 5.4. In certain embodiments, the second block can have a hydrogen HSP value of about 4 to about 9, about 4 to about 8, about 5 to about 8, or about 5 to about 7. Estimated Hansen Solubility Parameters can be computed using the HSPiP software package.

In certain embodiments, the second block has a glass transition temperature (Tg) value of less than about 100° C. In certain embodiments, the second block has a glass transition temperature (Tg) value of less than about 45° C. For example, the second block can have a Tg of about 25° C. to about 100° C., about 25° C. to about 80° C., about 25° C. to about 70° C., about 25° C. to about 60° C., about 25° C. to about 50° C., about 30° C. to about 50° C., or about 30° C. to about 45° C. Estimated glass transition temperature (Tg) can be computed using the HSPiP software package.

In certain embodiments, the second block has a surface energy value less than about 45 (e.g., about 25 to about 45). In certain embodiments, the second block has a surface energy value less than about 34. For example, in certain embodiments, the second block has a surface energy value of about 25 to about 34 or about 25 to about 31. In certain embodiments, the second block has a surface energy value greater than about 38. For example, in certain embodiments, the second block has a surface energy value of about 38 to about 40. In certain embodiments, the second block has a surface energy value of about 25 to about 40, about 28 to about 40, about 34 to about 40, or about 28 to about 35. Estimated surface energy values can be computed using the HSPiP software package.

In some embodiments, block copolymers as prepared herein can be described by the following structure:

CTACap-[first block]ₘ-[second block]ₙ-CTACap

where each CTACap is a capping unit derived from the chain transfer agent(s) used in the process for preparing the RAFT copolymer. The CTA used for preparing each of the first block and the second block can be the same or different. In some embodiments, the CTA used to prepare each the first block and the second block is the same (e.g., macroCTA). In some embodiments, the CTA used to prepare each of the first block and the second block is different. In some embodiments, the CTA used to prepare one or both of the first block and the second block comprises a functional group for the covalent attachment of a biomolecule, drug, or label to the block copolymer. In some embodiments, the covalent attachment can be via an ester or an amide bond. In some embodiments, the covalent attachment can be via EDC-NHS chemistry.

In some embodiments, the first block comprises a cap of formula:

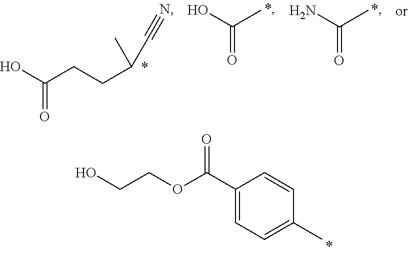

or a salt thereof, wherein * represents a point of covalent attachment to the first block. In certain preferred embodiments, the cap is

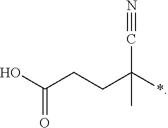

In some embodiments, the second capping unit is of formula

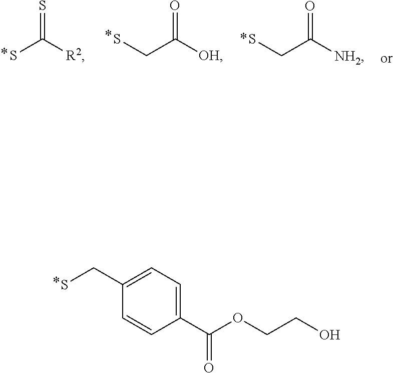

or a salt thereof, wherein * represents a point of covalent attachment to the second block, and R² is —SC₂-C₁₂ alkyl or C₆H₅ (i.e., phenyl), and is preferably

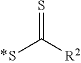

such as

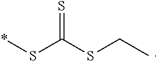 [SMILES: *SC(=S)SCC]

In some embodiments, a polymer nanoparticle comprises a block copolymer according to the present disclosure. In some embodiments, the block copolymer self-assembles into the nanoparticle.

In some embodiments, the diameter (e.g., a hydrodynamic diameter as measured by DLS) of the nanoparticle is about 15 nm to about 250 nm, about 15 nm to about 200 nm, about 15 nm to about 150 nm, about 20 nm to about 115 nm, about 20 nm to about 100 nm, about 20 nm to about 50 nm, or about 50 nm to about 115 nm.

In some embodiments, the nanoparticle has a molecular weight (as measured by NMR) of about 5 kDa to about 25 kDa, about 5 kDa to about 20 kDa, or about 10 kDa to about 20 kDa.

In some embodiments, a composition comprises a polymer nanoparticle as described herein and a nucleic acid (sometimes called a payload) complexed to the polymer nanoparticle. For example, the nucleic acid may be complexed to the nanoparticle through electrostatic interactions. In certain preferred embodiments, the polymer nanoparticle of the composition serves as a transfection agent to deliver a nucleic acid to a cell.

In certain embodiments, the nucleic acid is an RNA (e.g., an mRNA, a microRNA, antisense oligonucleotides, or an siRNA) or a DNA (e.g., an ssDNA, a dsDNA, or a complimentary coding DNA (cDNA), or a DNA plasmid (pDNA)). In certain embodiments, the nucleic acid is an mRNA. In certain embodiments, the nucleic acid is a circular RNA. In certain embodiments, the nucleic acid is a DNA plasmid (pDNA).

The polymer nanoparticles described herein are capable of interacting with (e.g., encapsulating or complexing with) nucleotide plasmids. In some embodiments, the encapsulation efficiency is greater than about 70%, greater than about 75%, greater than about 80%, greater than about 85%, greater than about 90%, greater than about 95%, or greater than about 97%. In certain embodiments, the encapsulation efficiency is about 80% to about 95%.

In certain embodiments, the diameter of the composition (i.e., the nanoparticle and the nucleic acid complexed to the nanoparticle) is similar to the dimeter of the nanoparticle before complexation (i.e., the diameter does not substantially increase upon complexation with the nucleic acid). In certain embodiments, the composition (i.e., the nanoparticle and the nucleic acid complex) has a diameter, and the diameter of the composition is less than about two-fold greater than the diameter of the nanoparticle without the nucleic acid. For example, the diameter of the composition may be less than about 1.5-fold greater or less than about 1.2 fold greater than the diameter of the nanoparticle without the nucleic acid being complexed. In certain embodiments, the diameter of the composition is about 1 fold to about 2 fold, about 1 fold to about 1.5 fold greater, or about 1 fold to about 1.2 fold greater than the diameter of the nanoparticle without the nucleic acid.

In some embodiments, the diameter of the composition (i.e., nanoparticle plus nucleotide such as RNA or DNA) compared to the diameter of the uncomplexed nanoparticle (e.g., the naked nanoparticle) is provided in fold change in size, as described in the examples. In some embodiments, the fold change is less than about 1.5 and may be about 0.1 to about 1.5, about 0.2 to about 1.5, about 0.2 to about 1, or about 0.2 to about 0.8.

In some embodiments, the diameter (e.g., a hydrodynamic diameter as measured by DLS) of the composition (i.e., the nanoparticle and the nucleotide (e.g., RNA such as mRNA) is about 20 nm to about 400 nm, about 100 nm to about 250 nm, about 20 nm to about 135 nm, about 30 nm to about 135 nm, or about 30 nm to about 100 nm.

In some embodiments, the diameter (e.g., a hydrodynamic diameter as measured by DLS) of the composition (i.e., the nanoparticle and the nucleotide (e.g., DNA such as pDNA) is about 20 nm to about 400 nm, about 75 nm to about 400 nm, about 150 nm to about 400 nm, about 150 nm to about 350 nm, or about 150 nm to about 250 nm.

In certain embodiments, the transfection efficiency is at least about 1%, or at least about 5%, or at least about 10% into cells, for example mammalian cells. In certain embodiments, the transfection efficiency is about 5% to about 35%, about 5% to about 30%, about 10% to about 30%, about 10% to about 25%, or about 15% to about 25%.

The nanoparticles described herein are capable of encapsulating nucleic acids. In some embodiments, the nucleic acid is from about 10 bp to about 25,000 bp. In some embodiments, the nucleic acid is at least 5 kbp or at least about 10 kbp. In some embodiments, the nucleic acid is about 10 bp to about 500 bp. In certain embodiments, the nucleic acid is about 10 kbp to about 25 kbp or about 10 kbp to about 15 kbp.

In certain embodiments, the ratio of nanoparticle to nucleic acid is about 5:1 to about 60:1 by weight. For example, the ratio of nanoparticle to nucleic acid may be about 10:1 or about 30:1 by weight.

In some embodiments, the polymer nanoparticles have low toxicity to cells. For example, in some embodiments, the cell viability after transfection of cells is at least about 70%, at least about 75%, at least about 80%, at least about 85%, at least about 90%, or at least about 95%.

In certain embodiments, the nanoparticle comprises a barcode construct covalently attached to the nanoparticle, as described in U.S. Patent Application Publication No. 2022/033309, the entirety of which is hereby incorporated by reference. As described in U.S. Patent Application Publication No. 2022/033309, the presence of the barcode construct can allow for identification of PNPs of interest, for example by determining the presence of a PNP in particular tissue or by performance in an assay. It should be understood that although certain examples in this application are performed on PNPs that include the barcode, similar performance would be expected by PNPs that lack the barcode.

The compositions described herein can be used for treating disease. For example, the polymer nanoparticles may be able to deliver a nucleic acid (i.e., a payload) that provides a therapeutic benefit to a patient. In certain embodiments, the polymer nanoparticle is able to deliver the nucleic acid to cells, such as neuronal cells. The compositions described herein can be used for treating disease, including neurological diseases (e.g., Parkinson's Disease or Alzheimer's Disease). In certain embodiments, the polymer nanoparticle delivers the nucleic acid to the brain or the cells of the central nervous system, for example microglia, astrocytes, oligodendrocytes, and neurons.

In one embodiment a composition comprising a polymer nanoparticle (e.g., a polymer nanoparticle derived from a controlled living/radical polymerization process, such as RAFT polymer) associated with a nucleic acid construct is provided. In another embodiment, a method of treating a patient with a disease is provided comprising administering to the patient the polymer nanoparticle.

In some embodiments, a method of treating a patient with a disease is provided, comprising administering to the patient the polymer nanoparticle identified in the in vivo screening method, wherein the polymer nanoparticle further comprises a drug payload, such as a polynucleotide or a protein payload, or a small molecule therapeutic or luminescent molecule payload, and treating the disease in the patient.

In various embodiments, any suitable route for administration of the library of polymer nanoparticles associated with nucleic acid constructs for the method of in vivo screening for the polymer nanoparticle associated with a nucleic acid construct, or for the method of treatment can be used including parenteral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In various embodiments, cell or tissue samples may be analyzed for the presence of the polymer nanoparticle associated with the nucleic acid constructs described herein. The samples can be any tissue, cell, or fluid sample from an animal, for example, selected from the group consisting of urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, lymph fluid, whole blood, serum, plasma, or any tissue or cell sample from an animal. Exemplary tissue or cell samples include brain tissue or cells, muscle tissue or cells, skin tissue or cells, heart tissue or cells, kidney tissue or cells, stomach tissue or cells, liver tissue or cells, urinary tract tissue or cells, gastrointestinal tract tissue or cells, head or neck tissue or cells, lung tissue or cells, reproductive tract tissue or cells, pancreatic tissue or cells, or any other tissue or cell type from an animal.

In one illustrative aspect for removing cells or tissues from the animal and isolating the nucleic acid constructs from the cells or tissues of the animal, the nucleic acid constructs are removed from cells or tissues of the animal. In various embodiments, nucleic acid constructs (e.g., DNA or RNA) obtained from the tissues or cells of the animal can be removed by rupturing the cells and isolating the nucleic acid constructs from the lysate. Techniques for rupturing cells and for isolation of nucleic acids are well-known in the art, and removal techniques include homogenization, such as by using a bead-beating technique. In other embodiments, the nucleic acid constructs may be isolated by rupturing cells using a detergent or a solvent, such as phenol-chloroform. In another aspect, the nucleic acid constructs may be separated from the lysate by physical methods including, but not limited to, centrifugation, dialysis, diafiltration, filtration, size exclusion, pressure techniques, digestion of proteins with Proteinase K, or by using a substance with an affinity for nucleic acids such as, for example, beads that bind nucleic acids.

In various embodiments, payloads may be combined with the polymer nanoparticles compositions using any or all of covalent bonds, electrostatic interactions, and ligand affinity interactions. In one aspect, covalent bonding methods include the use of EDC/NHS to form stable amide bonds between the payload and the polymer nanoparticles for improved stability (both “on the shelf” and in vivo), ease of separation and extraction, and sensitive detection. In another illustrative aspect, electrostatic bonding methods include the use of cationic polymer nanoparticles that electrostatically complex with the payload. In another embodiment, ligand affinity bonding includes the use of ligands such as avidin and biotin, both covalently bonded to the polymer nanoparticles and the payload via EDC/NHS chemistry to yield the stable combination of the payload and the polymer nanoparticles.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C(═S)S—R) between chains and the propagating radicals. The overall process is comprised of the insertion of monomers between the R— and Z—C(═S)S-groups of a RAFT agent (CTA), which form the α and β end-group of the majority of the resulting polymeric chains. Suitable CTAs for use in connection with the present disclosure include but are not limited to trithiocarbonates (Z=S-alkyl), dithiobenzoates (Z=Ph), dithiocarbamate (Z=N-alkyl), xanthates (Z=O-alkyl), and the like. (See, Sébastien Perrier, Macromolecules 2017 50 (19), 7433-7447). In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (˜pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis(carboxymethyl)trithiocarbonate, bis(2-amino-2-oxoethyl) trithiocarbonate, bis[4-(2-hydroxyethoxycarbonyl)benzyl]trithiocarbonate, 4-cyano-4-(ethylsulfanylthiocarbonyl) sulfanylvpentanoic acid, 4-cyano-4-((phenylcarbonothioyl)thio)pentanoic acid, and 4-cyano-4-[(dodecylsulfanylthiocarbonyl)sulfanyl]pentanoic acid, 4-cyano-4-(thiobenzoylthio)pentanoic acid, 2-cyano-2-propyl benzodithioate, cyanomethyl methyl(phenyl)carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

In some embodiments, the block copolymer can be associated with a DNA molecule, in particular a nucleic acid construct of the present disclosure, via several methods including, electrostatic interaction, high affinity, non-covalent bond, avidin-streptavidin conjugation, or by direct covalent attachment through, for example, an amide bond. In some embodiments, the RAFT copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via a high affinity, non-covalent bond, avidin-streptavidin conjugation. In some embodiments, the block copolymer can be associated with a DNA molecule, in particular a nucleic acid construct of the present disclosure, by direct covalent attachment through, for example, an amide bond.

The polymer nanoparticles described herein can be associated with a nucleic acid construct of the present disclosure via electrostatic interaction, avidin-streptavidin conjugation, or by direct covalent attachment. Exemplary interactions include: polymer nanoparticle (PNP) with positively charged corona in the case of electrostatic loading; nucleic acid constructs with negative charges due to the phosphate groups; electrostatically loaded PNP-nucleic acid construct complexes; carboxylate group on the terminal end of the polymer chains in the corona of the PNP; primary amine group on the 5′ end of the amine terminated nucleic acid construct; phosphate group on the 3′ end of the nucleic acid construct; amide bond formed in the direct amidification reaction between the amine terminal nucleic acid construct and the carboxylate terminated PNP; primary amine on the biotin bonding protein such as avidin; amide bond formed between the carboxylate group on the terminal end of the polymer chains in the corona of the PNP and the primary amine on the biotin bonding protein such as avidin; nucleic acid construct with a biotin functional group on the 5′ terminus; electrostatic coupling reaction that occurs when positively charged PNPs are mixed with negatively charged nucleic acid constructs; direct amidification reaction that is carried out via an EDA-NHC reaction between the carboxylate group on the terminal end of the polymer chains in the corona of the PNP and the primary amine on the amine terminated nucleic acid constructs; direct amidification reaction that is carried out via an EDA-NHC reaction between the carboxylate group on the terminal end of the polymer chains in the corona of the PNP and the primary amine on the biotin bonding protein such as avidin; coupling of the biotin on the 5′ end of the nucleic acid construct and the avidin conjugated to the carboxylate terminus on the corona of the PNPs.

In another illustrative embodiment, the polymer nanoparticle composition can be coated with one or more polymers to protect the compositions from immune responses or to enhance endosomal escape. In one embodiment, the one or more polymers used for coating comprise polyethylene glycol. In another embodiment, the one or more polymers used for coating comprise polyethylene glycol poly-L-lysine. In yet another embodiment, the one or more polymers used for coating comprise polyethylenimine. In an additional embodiment, the one or more polymers used for coating comprise polyethylene glycol poly-L-lysine and polyethylenimine.

It will be appreciated that tuning the parameters and properties of the block copolymers described herein can be advantageous to their use in the compositions and methods as described herein. Accordingly, the methods for preparing block copolymers either in singleton or in library format as described herein are capable of providing particular parameters and properties of the block copolymers.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), HexaneIsobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, t-Butanol, Tetrahydrofuran, Trifluoroacetic acid, water, and the like, and combinations thereof.

In certain embodiments, a soluble/dispersible polymer nanoparticle have diameters ranging from ˜20 nm to ˜110 nm) when tested alone and maintained small diameters (˜35 nm to ˜125 nm) when efficiently loaded with mRNA (˜85-90% loading efficiency).

In certain embodiments, a set of polymer nanoparticle (PNP) design parameters and reversible addition-fragmentation chain transfer (RAFT) polymerization conditions produced PNPs with small diameters (ranging from ˜20 nm to ˜110 nm) when tested alone and maintain small diameters (˜35 nm to ˜125 nm) when efficiently loaded with mRNA (˜85-90% loading efficiency determined via Ribogreen assay). Certain PNP design parameters include:
1) Cationic p(DMAEMA) macro-Chain Transfer Agents (mCTAs or Block Is) with theoretical degrees of polymerization (DP) values of 50, 75, 100, and 125 resulted in measured molecular weights of 5,456 kDa, 7,728 kDa, 9,900 kDa, and 10,314 kDa, respectively. These p(DMAEMA) mCTAs may facilitate electrostatic binding with negatively charged mRNA.
2) Hydrophobic monomer butyl methacrylate (BMA) may be used at a target of 60% theoretical molar composition in the PNP core block (Block 2) to stabilize the PNP, improve cytocompatibility, and help membrane interactions putatively needed for endosomal escape upon PNP destabilization following exposure to decreased pH levels in the endosomal pathway.
3) pH-responsive ethyl acrylic acid (EAA) or propyl acrylic acid (PAA) monomers at, for example, a 40% theoretical molar composition within the PNP core block (Block 2) provide improved endosomolytic activity which may be due to a “proton-sponge effect” upon transfection within cells, whereby water content in endosomes increases to the point where the internal pressure causes endosomal membrane disruption, thus releasing the PNPs and mRNA into the cytoplasm to facilitate successful translation of functional proteins. Increased carbon side chain lengths of EAA and PAA may help the hydrophobicity (i.e., stability) of these PNPs since recipes without acrylic acids or containing a target of 40% methyl acrylic acid (MAA) did not maintain small diameters upon mRNA loading.
4) Block 2 theoretical DP values of 20, 30, 40, or 50.
5) 24 hr reaction in DMF under Ar @70 C.

### ALTERNATIVE EMBODIMENTS

1. A block copolymer comprising
   a first block having a monomer unit represented by formula I:

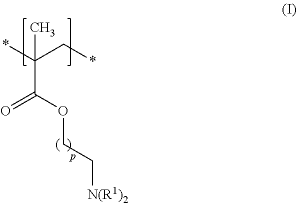

or a salt thereof, wherein:
p is 0 or an integer selected from 1-3,
each R¹ is individually selected from H or alkyl; and
wherein each * represents a connecting point to the rest of the block copolymer; and
a copolymer second block comprising a copolymer of monomer units selected from formula II:

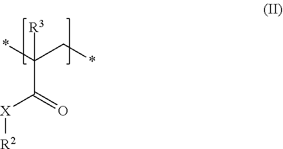

or a salt thereof, wherein:
X is —O—;
R² is alkyl; and
R³ is alkyl;
wherein each * represents a connecting point to the rest of the block copolymer; and formula III:

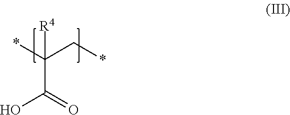

or a salt thereof, wherein:
   R⁴ is alkyl;
wherein each * represents a connecting point to the rest of the block copolymer;
wherein second block has a C Log P greater than about 1.5.
2. The block copolymer of embodiment 1, wherein p is 1.
3. The block copolymer of embodiment 1 or 2, wherein each R¹ is C₁-C₃ alkyl.
4. The block copolymer of embodiment 1 or 2, wherein each R¹ is methyl.
5. The block copolymer of any one of embodiments 1-4, wherein R² is C₁-C₆ alkyl.
6. The block copolymer of any one of embodiments 1-5, wherein R² is butyl.
7. The block copolymer of any one of embodiments 1-6, wherein R³ is C₁-C₆ alkyl.
8. The block copolymer of any one of embodiments 1-7, wherein R³ is methyl.
9. The block copolymer of any one of embodiments 1-8, wherein R⁴ is C₁-C₆ alkyl.
10. The block copolymer of any one of embodiments 1-9, wherein R⁴ is ethyl or propyl.
11. The block copolymer of any one of embodiments 1-10, wherein R⁴ is ethyl.
12. The block copolymer of any one of embodiments 1-10, wherein R⁴ is propyl.
13. A block copolymer comprising a first block and a second block, wherein:
   the first block comprise a homopolymer of poly dimethylaminoethyl methacrylate (DMAEMA), and
   the second block comprises a copolymer of an alkylacrylate (e.g., butylmethacrylate (BMA)) and an acrylic acid (e.g., ethylacrylic acid (EAA) or propylacrylic acid (PAA));
   wherein the first block is about 40% to about 80% by weight of the block copolymer.
14. The block copolymer of any one of the preceding embodiments, wherein the first block has a molecular weight of about 4,000 Da to about 15,000 Da.
15. The block copolymer of any one of the preceding embodiments, wherein the first block has a molecular weight of about 4,000 Da to about 13,000 Da.
16. The block copolymer of any one of the preceding embodiments, wherein the first block has a molecular weight of about 5,000 Da to about 10,000 Da.
17. The block copolymer of any one of the preceding embodiments, wherein the copolymer has a total molecular weight of about 5 kDa to about 25 kDa.
18. The block copolymer of any one of the preceding embodiments, wherein the copolymer has a total molecular weight of about 5 kDa to about 20 kDa.
19. The block copolymer of any one of the preceding embodiments, wherein the first block has a degree of polymerization of about 30 to about 150.
20. The block copolymer of any one of the preceding embodiments, wherein the second block has a degree of polymerization of about 15 to about 60.
21. The block copolymer of any one of the preceding embodiments, wherein the second block has a degree of polymerization of about 15 to about 25.
22. The block copolymer of any one of embodiments 1-20, wherein the second block has a degree of polymerization of about 25 to about 35.
23. The block copolymer of any one of embodiments 1-20, wherein the second block has a degree of polymerization of about 35 to about 45.
24. The block copolymer of any one of embodiments 1-20, wherein the second block has a degree of polymerization of about 45 to about 55.
25. The block copolymer of any one of the preceding embodiments, wherein the second block has a molecular weight of about 1 kDa to about 13 kDa.
26. The block copolymer of any one of embodiments 1-24, wherein the second block has a molecular weight of about 2 kDa to about 5 kDa.
27. The block copolymer of any one of embodiments 1-24, wherein the second block has a molecular weight of about 2 kDa to about 4 kDa.
28. The block copolymer of any one of embodiments 1-24, wherein the second block has a molecular weight of about 3 kDa to about 5 kDa.
29. The block copolymer of any one of the preceding embodiments, wherein the second block has a C Log P of at least 1.6.
30. The block copolymer of any one of the preceding embodiments, wherein the second block has a C Log P of at least 1.7.
31. The block copolymer of any one embodiments 1-29, wherein the second block has a C Log P of about 1.5 to about 3.
32. The block copolymer of any one of embodiments 1-29, wherein the second block has a C Log P of about 1.5 to about 2.6.
33. The block copolymer of embodiments 1-29, wherein the second block has a C Log P of about 1.6 to about 2.6.
34. The block copolymer of any one of the preceding embodiments, wherein the molar ratio of the molecular weights of the compound of formula II and the compound of formula III in the second block is about 30:70 to about 90:10.
35. The block copolymer of any one of the preceding embodiments, wherein the ratio of the molecular weights of the compound of formula II and the compound of formula III in the second block is about 50:50 to about 75:25.
36. The block copolymer of any one of the preceding embodiments, wherein the ratio of the molecular weights of the compound of formula II and the compound of formula III in the second block is about 60:40.
37. The block copolymer of any one of the preceding embodiments, wherein the first block comprises a cap of formula:

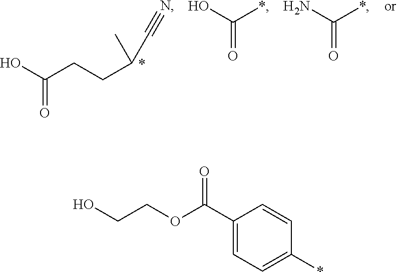

or a salt thereof, wherein * represents a point of covalent attachment to the first block.
38. The block copolymer of any one of the preceding embodiments, wherein the first block comprises a cap of formula:

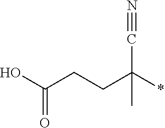 [SMILES: *C(C)(C#N)CCC(=O)O]

or a salt thereof, wherein the * represents a point of connection to the first block.
39. The block copolymer of any one of the preceding embodiments, wherein the second block comprises a cap of formula:

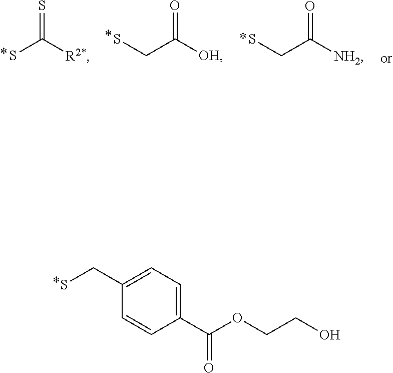

or a salt thereof, wherein * represents a point of covalent attachment to the second block, and R²* is —SC₂-C₁₂ alkyl or C₆H.
40. The block copolymer of any one of the preceding embodiments, wherein the second block comprises a cap of formula:

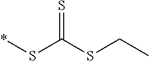 [SMILES: *SC(=S)SCC]

or a salt thereof, wherein the * represents a point of connection to the second block.
41. A polymer nanoparticle comprising:
   the block copolymer according to any one of embodiments 1-40,
   wherein the nanoparticle has a diameter of about 15 nm to about 250 nm.
42. The polymer nanoparticle of embodiment 41, wherein the nanoparticle has a diameter of about 20 nm to about 115 nm.
43. The polymer nanoparticle of embodiment 41, wherein the nanoparticle has a diameter of about 20 nm to about 100 nm.
44. The polymer nanoparticle of embodiment 41, wherein the nanoparticle has a diameter of about 20 nm to about 50 nm.
45. The polymer nanoparticle of embodiment 41, wherein the nanoparticle has a diameter of about 50 nm to about 115 nm.
46. A composition comprising:
   a polymer nanoparticle according to any one of embodiments 41-45, and
   a nucleic acid complexed to the polymer nanoparticle.
47. The composition of embodiment 46, wherein the polymer nanoparticle is complexed to the nucleic acid via electrostatic interaction.
48. The composition according to embodiment 46 or 47, wherein the composition has a diameter, and the diameter of the composition is less than about two-fold greater than the diameter of the nanoparticle without the nucleic acid.
49. The composition according to embodiment 46 or 47, wherein the composition has a diameter, and the diameter of the composition is less than about 1.5-fold greater than the diameter of the nanoparticle without the nucleic acid.
50. The composition according to embodiment 46 or 47, wherein the composition has a diameter, and the diameter of the composition is less than about 1.2-fold greater than the diameter of the nanoparticle without the nucleic acid.
51. The composition according to embodiment 46 or 47, wherein the composition has a diameter, and the diameter of the composition is about 1 fold to about 2 fold greater than the diameter of the nanoparticle without the nucleic acid.
52. The composition according to embodiment 46 or 47, wherein the composition has a diameter, and the diameter of the composition is about 1 fold to about 1.5 fold greater than the diameter of the nanoparticle without the nucleic acid.
53. The composition according to embodiment 46 or 47, wherein the composition has a diameter, and the diameter of the composition is about 1 fold to about 1.2 fold greater than the diameter of the nanoparticle without the nucleic acid.
54. The composition of any one of embodiments 46-53, wherein the nucleic acid is an RNA (e.g., mRNA).
55. The composition of any one of embodiments 46-53, wherein the nucleic acid is a DNA (e.g., pDNA).
56. The composition of any one of embodiments 46-55, wherein the percent encapsulation is at least about 80%.
57. The composition of any one of embodiments 46-55, wherein the percent encapsulation is at least about 80% to about 95%.
58. The composition of any one of embodiments 46-57, wherein the ratio of nucleic acid to nanoparticle is about 5:1 to about 60:1.
59. The composition of any one of embodiments 46-57, wherein the ratio of nucleic acid to nanoparticle is about 10:1 to about 60:1.
60. The composition of any one of embodiments 46-57, wherein the ratio of nucleic acid to nanoparticle is about 10:1 to about 30:1.
61. The composition of any one of embodiments 46-57, further comprising a pharmaceutically acceptable carrier.
62. A method of treating a disease (e.g., a neurological disease) in a patient in need thereof, the method comprising:
   administering a therapeutically effective amount of a composition according to any one of embodiments 46-61.
63. A method of transfecting a cell, the method comprising:
   contacting the cell with an effective amount of a composition according to any one of embodiments 46-61.
64. The method of embodiment 63, wherein the method is in vitro.
65. The method of embodiment 63, wherein the method is in vivo.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

### EXAMPLES

### Polymer Nanoparticle Synthesis

Diblock copolymers were synthesized similarly as described in U.S. Patent Application Publication No. 2022/0175812, the entirety of which is incorporated by reference herein, with some modifications using reversible addition-fragmentation chain transfer (RAFT) polymerization with reagents and amounts listed in Table 2. Block 1 reagents, including 2-(N,N-Dimethylamino)ethyl methacrylate (DMAEMA) as the monomer, 4-Cyano-4-[(ethylsulfanylthiocarbonyl)sulfanyl]pentanoic acid (ECT) as the chain transfer agent (CTA), 2,2′-Azobis(2-methylpropionitrile (AIBN) as the initiator, and N,N-Dimethylformamide (DMF) as the solvent, were combined in a 100 mL round-bottom flasks, purged with argon, and heated to 60° C. for 24 hours using an oil bath. The reaction products were purified using four 80:20 pentane:ether precipitation washes and centrifugation cycles and dried in vacuo. The Block 1 products were characterized for molecular weight using GPC, and used as the macroRAFT agents for Block 2, and the calculated reagent volumes (as calculated based on theoretical molecular weight information for Block 1) were combined with AIBN initiator and various acrylate monomers in Corning 96-well polypropylene cluster tubes for the Block 2 reactions. The reaction mixtures were argon purged before being heated at 70° C. for 24 hours. The reaction products were purified using either four 80:20 pentane:ether precipitation washes and centrifugation cycles or three 80:20 pentane:diethyl ether washes and centrifugation cycles followed by drying in vacuo. Finally, the purified materials were lyophilized for 3-4 days and stored at room temperature for experimental use.

Block copolymers were prepared using Reversible Addition-Fragmentation Chain Transfer (RAFT) polymerization, using the monomers shown in Table 1 below. Block copolymers included:
p(DMAEMA)-b-p(MMA-co-MAA),
p(DMAEMA)-b-p(MMA-co-EAA),
p(DMAEMA)-b-p(MMA-co-PAA),
p(DMAEMA)-b-p(BMA-co-MAA),
p(DMAEMA)-b-p(BMA-co-EAA), and
p(DMAEMA)-b-p(BMA-co-PAA)
   with block 1 degree of polymerization ranging from 30 to 70 (MW range from 5 to 11 kDa), and block 2 degree of polymerization ranging from 20 to 150 (molecular weight range from 7 to 26 kDas). A total of 221 polymers were synthesized and characterized using the materials and the methods described above. The polymer compositions described in Table 2 include Block 1 molecular weights from 5 to 50 kDa, Block 1 degree of polymerization from 35 to 315, Block 2 molecular weights from 1 to 16 kDa, Block 2 Degrees of polymerization from 8 to 159, Block 2 MMA or BMA molar compositions of 18 to 90 mol %, and block 2 EAA or PAA compositions from 10 to 82 mol %.

TABLE 1
Monomers used in the nanoparticle forming polymers (PNPs). XLogP3 values are those
reported in PubChem, and are used to calculate molar weighted average CLogP values of polymer blocks.

| Monomer Name | Abbreviation | CAS | Structure | XLogP3 |
|---|---|---|---|---|
| Methacrylic Acid | MAA | 79-41-4 | 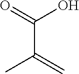 | 0.9 |
| Ethylacrylic acid | EAA | 3586-58-1 | 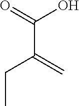 | 1.2 |
| Propylacrylic acid | PAA | 5650-75-9 | 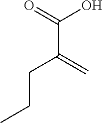 | 1.7 |
| Methyl methacrylate | MMA | 80-62-6 | 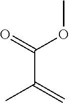 | 1.4 |
| Butyl methacrylate | BMA | 97-88-1 | 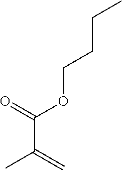 | 2.9 |
| Dimethylaminoethyl methacrylate | DMAEMA | 2867-47-2 | 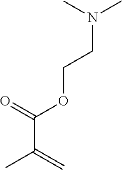 | 1.2 |

TABLE 2
PNP composition

| | Block 1 Composition | | | Block 2 Composition | | | | | |
|---|---|---|---|---|---|---|---|---|---|
| PNP | | mCTA | | | Mol | | Mol | | B2 |
| ID | B1 | Mw | DP B1 | Monomer | % | Monomer | % | DP B2 | Mw |
| ID | Monomer | (kDa) | Overall | 1 | B2M1 | 2 | B2M2 | Overall | (kDa) |
| 1 | DMAEMA | 5.5 | 35 | MMA | 83 | MAA | 17 | 15 | 1.5 |
| 2 | DMAEMA | 7.7 | 49 | MMA | 75 | MAA | 25 | 24 | 2.3 |
| 3 | DMAEMA | 9.9 | 63 | MMA | 78 | MAA | 22 | 22 | 2.1 |
| 4 | DMAEMA | 10.3 | 66 | MMA | 55 | MAA | 45 | 33 | 3.1 |
| 5 | DMAEMA | 5.5 | 35 | MMA | 19 | EAA | 81 | 44 | 4.4 |
| 6 | DMAEMA | 7.7 | 49 | MMA | 22 | EAA | 78 | 71 | 7.1 |
| 7 | DMAEMA | 9.9 | 63 | MMA | 27 | EAA | 73 | 95 | 9.5 |
| 8 | DMAEMA | 10.3 | 66 | MMA | 31 | EAA | 69 | 116 | 11.6 |
| 9 | DMAEMA | 5.5 | 35 | MMA | 65 | PAA | 35 | 12 | 1.3 |
| 10 | DMAEMA | 7.7 | 49 | MMA | 72 | PAA | 28 | 30 | 3.2 |
| 11 | DMAEMA | 9.9 | 63 | MMA | 67 | PAA | 33 | 41 | 4.3 |
| 12 | DMAEMA | 10.3 | 66 | MMA | 75 | PAA | 25 | 48 | 5.0 |
| 13 | DMAEMA | 5.5 | 35 | BMA | 88 | MAA | 12 | 18 | 2.5 |
| 14 | DMAEMA | 7.7 | 49 | BMA | 90 | MAA | 10 | 31 | 4.2 |
| 15 | DMAEMA | 5.5 | 35 | BMA | 24 | EAA | 76 | 48 | 5.2 |
| 16 | DMAEMA | 7.7 | 49 | BMA | 25 | EAA | 75 | 72 | 7.9 |
| 17 | DMAEMA | 9.9 | 63 | BMA | 25 | EAA | 75 | 95 | 10.5 |
| 18 | DMAEMA | 10.3 | 66 | BMA | 29 | EAA | 71 | 100 | 11.3 |
| 19 | DMAEMA | 5.5 | 35 | BMA | 65 | PAA | 35 | 17 | 2.2 |
| 20 | DMAEMA | 7.7 | 49 | BMA | 68 | PAA | 32 | 25 | 3.3 |
| 21 | DMAEMA | 9.9 | 63 | BMA | 65 | PAA | 35 | 35 | 4.6 |
| 22 | DMAEMA | 9.9 | 63 | MMA | 44 | MAA | 56 | 29 | 2.7 |
| 23 | DMAEMA | 9.9 | 63 | MMA | 35 | EAA | 65 | 43 | 4.3 |
| 24 | DMAEMA | 10.3 | 66 | MMA | 29 | EAA | 71 | 93 | 9.3 |
| 25 | DMAEMA | 9.9 | 63 | MMA | 44 | EAA | 56 | 140 | 14.0 |
| 26 | DMAEMA | 10.3 | 66 | MMA | 44 | EAA | 56 | 159 | 15.9 |
| 27 | DMAEMA | 9.9 | 63 | MMA | 65 | PAA | 35 | 41 | 4.3 |
| 28 | DMAEMA | 10.3 | 66 | MMA | 66 | PAA | 34 | 55 | 5.8 |
| 29 | DMAEMA | 9.9 | 63 | MMA | 56 | PAA | 44 | 49 | 5.2 |
| 30 | DMAEMA | 5.5 | 35 | BMA | 26 | EAA | 74 | 15 | 1.7 |
| 31 | DMAEMA | 7.7 | 49 | BMA | 18 | EAA | 82 | 25 | 2.6 |
| 32 | DMAEMA | 5.5 | 35 | BMA | 19 | EAA | 81 | 25 | 2.7 |
| 33 | DMAEMA | 7.7 | 49 | BMA | 23 | EAA | 77 | 32 | 3.5 |
| 34 | DMAEMA | 5.5 | 35 | BMA | 25 | EAA | 75 | 21 | 2.3 |
| 35 | DMAEMA | 7.7 | 49 | BMA | 30 | EAA | 70 | 30 | 3.4 |
| 36 | DMAEMA | 5.5 | 35 | BMA | 32 | EAA | 68 | 22 | 2.5 |
| 37 | DMAEMA | 7.7 | 49 | BMA | 36 | EAA | 64 | 37 | 4.3 |
| 38 | DMAEMA | 5.5 | 35 | BMA | 30 | EAA | 70 | 32 | 3.6 |
| 39 | DMAEMA | 7.7 | 49 | BMA | 31 | EAA | 69 | 40 | 4.5 |
| 40 | DMAEMA | 5.5 | 35 | BMA | 39 | EAA | 61 | 33 | 3.8 |
| 41 | DMAEMA | 7.7 | 49 | BMA | 26 | EAA | 74 | 34 | 3.8 |
| 42 | DMAEMA | 5.5 | 35 | BMA | 44 | EAA | 56 | 32 | 3.8 |
| 43 | DMAEMA | 7.7 | 49 | BMA | 44 | EAA | 56 | 44 | 5.2 |
| 44 | DMAEMA | 5.5 | 35 | BMA | 50 | EAA | 50 | 40 | 4.8 |
| 45 | DMAEMA | 7.7 | 49 | BMA | 49 | EAA | 51 | 50 | 6.0 |
| 46 | DMAEMA | 5.5 | 35 | BMA | 45 | PAA | 55 | 8 | 1.0 |
| 47 | DMAEMA | 7.7 | 49 | BMA | 44 | PAA | 56 | 11 | 1.4 |
| 48 | DMAEMA | 5.5 | 35 | BMA | 54 | PAA | 46 | 8 | 1.0 |
| 49 | DMAEMA | 7.7 | 49 | BMA | 88 | PAA | 12 | 22 | 3.0 |
| 50 | DMAEMA | 5.5 | 35 | BMA | 47 | PAA | 53 | 11 | 1.4 |
| 51 | DMAEMA | 7.7 | 49 | BMA | 43 | PAA | 57 | 18 | 2.2 |
| 52 | DMAEMA | 5.5 | 35 | BMA | 57 | PAA | 43 | 12 | 1.6 |
| 53 | DMAEMA | 7.7 | 49 | BMA | 66 | PAA | 34 | 23 | 3.0 |
| 54 | DMAEMA | 5.5 | 35 | BMA | 51 | PAA | 49 | 17 | 2.2 |
| 55 | DMAEMA | 7.7 | 49 | BMA | 44 | PAA | 56 | 34 | 4.3 |
| 56 | DMAEMA | 5.5 | 35 | BMA | 68 | PAA | 32 | 19 | 2.6 |
| 57 | DMAEMA | 7.7 | 49 | BMA | 71 | PAA | 29 | 25 | 3.4 |
| 58 | DMAEMA | 5.5 | 35 | BMA | 55 | PAA | 45 | 22 | 2.8 |
| 59 | DMAEMA | 7.7 | 49 | BMA | 53 | PAA | 47 | 21 | 2.8 |
| 60 | DMAEMA | 5.5 | 35 | BMA | 71 | PAA | 29 | 27 | 3.6 |
| 61 | DMAEMA | 7.7 | 49 | BMA | 72 | PAA | 28 | 38 | 5.1 |
| 62 | DMAEMA | 7.4 | 47 | BMA | 40 | EAA | 60 | 30 | 3.5 |
| 63 | DMAEMA | 8.0 | 51 | BMA | 45 | EAA | 55 | 23 | 2.7 |
| 64 | DMAEMA | 8.7 | 55 | BMA | 22 | EAA | 78 | 34 | 3.7 |
| 65 | DMAEMA | 9.9 | 63 | BMA | 31 | EAA | 69 | 24 | 2.8 |
| 66 | DMAEMA | 7.4 | 47 | BMA | 39 | EAA | 61 | 34 | 4.0 |
| 67 | DMAEMA | 8.0 | 51 | BMA | 33 | EAA | 67 | 43 | 4.9 |
| 68 | DMAEMA | 8.7 | 55 | BMA | 27 | EAA | 73 | 38 | 4.2 |
| 69 | DMAEMA | 9.9 | 63 | BMA | 32 | EAA | 68 | 34 | 3.8 |
| 70 | DMAEMA | 7.4 | 47 | BMA | 42 | EAA | 58 | 43 | 5.0 |
| 71 | DMAEMA | 8.0 | 51 | BMA | 37 | EAA | 63 | 43 | 4.9 |
| 72 | DMAEMA | 8.7 | 55 | BMA | 36 | EAA | 64 | 43 | 5.0 |
| 73 | DMAEMA | 9.9 | 63 | BMA | 29 | EAA | 71 | 45 | 5.1 |
| 74 | DMAEMA | 7.4 | 47 | BMA | 39 | EAA | 61 | 46 | 5.3 |
| 75 | DMAEMA | 8.0 | 51 | BMA | 51 | EAA | 49 | 45 | 5.4 |
| 76 | DMAEMA | 8.7 | 55 | BMA | 39 | EAA | 61 | 45 | 5.2 |
| 77 | DMAEMA | 9.9 | 63 | BMA | 38 | EAA | 62 | 40 | 4.7 |
| 78 | DMAEMA | 7.4 | 47 | BMA | 62 | PAA | 38 | 14 | 1.9 |
| 79 | DMAEMA | 8.0 | 51 | BMA | 61 | PAA | 39 | 16 | 2.1 |
| 80 | DMAEMA | 8.7 | 55 | BMA | 58 | PAA | 42 | 15 | 1.9 |
| 81 | DMAEMA | 9.9 | 63 | BMA | 61 | PAA | 39 | 13 | 1.7 |
| 82 | DMAEMA | 7.4 | 47 | BMA | 68 | PAA | 32 | 24 | 3.2 |
| 83 | DMAEMA | 8.0 | 51 | BMA | 63 | PAA | 37 | 20 | 2.7 |
| 84 | DMAEMA | 8.7 | 55 | BMA | 57 | PAA | 43 | 15 | 1.9 |
| 85 | DMAEMA | 9.9 | 63 | BMA | 63 | PAA | 37 | 16 | 2.1 |
| 86 | DMAEMA | 7.4 | 47 | BMA | 64 | PAA | 36 | 24 | 3.2 |
| 87 | DMAEMA | 8.0 | 51 | BMA | 64 | PAA | 36 | 23 | 3.0 |
| 88 | DMAEMA | 8.7 | 55 | BMA | 65 | PAA | 35 | 21 | 2.7 |
| 89 | DMAEMA | 9.9 | 63 | BMA | 58 | PAA | 42 | 18 | 2.3 |
| 90 | DMAEMA | 7.4 | 47 | BMA | 64 | PAA | 36 | 25 | 3.4 |
| 91 | DMAEMA | 8.0 | 51 | BMA | 66 | PAA | 34 | 26 | 3.4 |
| 92 | DMAEMA | 8.7 | 55 | BMA | 64 | PAA | 36 | 22 | 2.9 |
| 93 | DMAEMA | 9.9 | 63 | BMA | 62 | PAA | 38 | 21 | 2.8 |
| 94 | DMAEMA | 8.0 | 51 | BMA | 27 | EAA | 73 | 34 | 3.8 |
| 95 | DMAEMA | 8.7 | 55 | BMA | 27 | EAA | 73 | 34 | 3.8 |
| 96 | DMAEMA | 8.0 | 51 | BMA | 26 | EAA | 74 | 39 | 4.3 |
| 97 | DMAEMA | 8.7 | 55 | BMA | 30 | EAA | 70 | 36 | 4.1 |
| 98 | DMAEMA | 8.0 | 51 | BMA | 30 | EAA | 70 | 42 | 4.7 |
| 99 | DMAEMA | 8.7 | 55 | BMA | 27 | EAA | 73 | 42 | 4.7 |
| 100 | DMAEMA | 8.0 | 51 | BMA | 31 | EAA | 69 | 42 | 4.7 |
| 101 | DMAEMA | 8.7 | 55 | BMA | 27 | EAA | 73 | 45 | 5.0 |
| 102 | DMAEMA | 8.0 | 51 | BMA | 36 | EAA | 64 | 44 | 5.1 |
| 103 | DMAEMA | 8.7 | 55 | BMA | 32 | EAA | 68 | 47 | 5.3 |
| 104 | DMAEMA | 8.0 | 51 | BMA | 36 | EAA | 64 | 48 | 5.5 |
| 105 | DMAEMA | 8.7 | 55 | BMA | 32 | EAA | 68 | 49 | 5.5 |
| 106 | DMAEMA | 8.0 | 51 | BMA | 36 | EAA | 64 | 51 | 5.9 |
| 107 | DMAEMA | 8.7 | 55 | BMA | 38 | EAA | 62 | 50 | 5.8 |
| 108 | DMAEMA | 8.0 | 51 | BMA | 40 | EAA | 60 | 47 | 5.5 |
| 109 | DMAEMA | 8.7 | 55 | BMA | 37 | EAA | 63 | 53 | 6.1 |
| 110 | DMAEMA | 8.0 | 51 | BMA | 55 | PAA | 45 | 15 | 2.0 |
| 111 | DMAEMA | 8.7 | 55 | BMA | 56 | PAA | 44 | 15 | 2.0 |
| 112 | DMAEMA | 8.0 | 51 | BMA | 63 | PAA | 37 | 15 | 2.0 |
| 113 | DMAEMA | 8.7 | 55 | BMA | 60 | PAA | 40 | 16 | 2.1 |
| 114 | DMAEMA | 8.0 | 51 | BMA | 58 | PAA | 42 | 20 | 2.6 |
| 115 | DMAEMA | 8.7 | 55 | BMA | 60 | PAA | 40 | 18 | 2.4 |
| 116 | DMAEMA | 8.0 | 51 | BMA | 62 | PAA | 38 | 18 | 2.4 |
| 117 | DMAEMA | 8.7 | 55 | BMA | 64 | PAA | 36 | 19 | 2.5 |
| 118 | DMAEMA | 8.0 | 51 | BMA | 61 | PAA | 39 | 24 | 3.1 |
| 119 | DMAEMA | 8.7 | 55 | BMA | 59 | PAA | 41 | 21 | 2.7 |
| 120 | DMAEMA | 8.0 | 51 | BMA | 64 | PAA | 36 | 22 | 2.9 |
| 121 | DMAEMA | 8.7 | 55 | BMA | 65 | PAA | 35 | 21 | 2.8 |
| 122 | DMAEMA | 8.0 | 51 | BMA | 60 | PAA | 40 | 26 | 3.3 |
| 123 | DMAEMA | 8.7 | 55 | BMA | 63 | PAA | 37 | 26 | 3.4 |
| 124 | DMAEMA | 8.0 | 51 | BMA | 66 | PAA | 34 | 25 | 3.3 |
| 125 | DMAEMA | 8.7 | 55 | BMA | 63 | PAA | 37 | 25 | 3.3 |
| 126 | DMAEMA | 19.2 | 122 | BMA | 51 | PAA | 49 | 33 | 4.3 |
| 127 | DMAEMA | 19.2 | 122 | BMA | 51 | PAA | 49 | 37 | 4.8 |
| 128 | DMAEMA | 36.6 | 233 | BMA | 44 | PAA | 56 | 37 | 4.7 |
| 129 | DMAEMA | 36.6 | 233 | BMA | 45 | PAA | 55 | 40 | 5.1 |
| 130 | DMAEMA | 49.6 | 315 | BMA | 41 | PAA | 59 | 34 | 4.3 |
| 131 | DMAEMA | 49.6 | 315 | BMA | 28 | PAA | 72 | 35 | 4.2 |
| 132 | DMAEMA | 49.6 | 315 | BMA | 33 | PAA | 67 | 36 | 4.5 |
| 133 | DMAEMA | 49.6 | 315 | BMA | 39 | PAA | 61 | 40 | 5.1 |
| 134 | DMAEMA | 45.5 | 289 | BMA | 43 | PAA | 57 | 37 | 4.7 |
| 135 | DMAEMA | 45.5 | 289 | BMA | 43 | PAA | 57 | 37 | 4.7 |
| 136 | DMAEMA | 45.5 | 289 | BMA | 39 | PAA | 61 | 29 | 3.7 |
| 137 | DMAEMA | 45.5 | 289 | BMA | 40 | PAA | 60 | 41 | 5.1 |
| 138 | DMAEMA | 19.2 | 122 | BMA | 56 | PAA | 44 | 37 | 4.8 |
| 139 | DMAEMA | 19.2 | 122 | BMA | 57 | PAA | 43 | 38 | 5.0 |
| 140 | DMAEMA | 36.6 | 233 | BMA | 47 | PAA | 53 | 43 | 5.5 |
| 141 | DMAEMA | 36.6 | 233 | BMA | 54 | PAA | 46 | 50 | 6.5 |
| 142 | DMAEMA | 49.6 | 315 | BMA | 46 | PAA | 54 | 52 | 6.6 |
| 143 | DMAEMA | 49.6 | 315 | BMA | 47 | PAA | 53 | 53 | 6.8 |
| 144 | DMAEMA | 49.6 | 315 | BMA | 49 | PAA | 51 | 57 | 7.3 |
| 145 | DMAEMA | 49.6 | 315 | BMA | 47 | PAA | 53 | 58 | 7.4 |
| 146 | DMAEMA | 45.5 | 289 | BMA | 50 | PAA | 50 | 54 | 6.9 |
| 147 | DMAEMA | 45.5 | 289 | BMA | 48 | PAA | 52 | 54 | 6.9 |
| 148 | DMAEMA | 45.5 | 289 | BMA | 47 | PAA | 53 | 51 | 6.5 |
| 149 | DMAEMA | 45.5 | 289 | BMA | 47 | PAA | 53 | 49 | 6.2 |
| 150 | DMAEMA | 19.2 | 122 | BMA | 62 | PAA | 38 | 42 | 5.5 |
| 151 | DMAEMA | 19.2 | 122 | BMA | 62 | PAA | 38 | 43 | 5.6 |
| 152 | DMAEMA | 36.6 | 233 | BMA | 48 | PAA | 52 | 51 | 6.5 |
| 153 | DMAEMA | 36.6 | 233 | BMA | 54 | PAA | 46 | 52 | 6.7 |
| 154 | DMAEMA | 49.6 | 315 | BMA | 45 | PAA | 55 | 58 | 7.4 |
| 155 | DMAEMA | 49.6 | 315 | BMA | 50 | PAA | 50 | 64 | 8.2 |
| 156 | DMAEMA | 49.6 | 315 | BMA | 48 | PAA | 52 | 50 | 6.4 |
| 157 | DMAEMA | 49.6 | 315 | BMA | 41 | PAA | 59 | 54 | 6.8 |
| 158 | DMAEMA | 45.5 | 289 | BMA | 49 | PAA | 51 | 60 | 7.7 |
| 159 | DMAEMA | 45.5 | 289 | BMA | 47 | PAA | 53 | 48 | 6.2 |
| 160 | DMAEMA | 45.5 | 289 | BMA | 46 | PAA | 54 | 50 | 6.4 |
| 161 | DMAEMA | 45.5 | 289 | BMA | 49 | PAA | 51 | 46 | 5.9 |
| 162 | DMAEMA | 19.2 | 122 | BMA | 59 | PAA | 41 | 40 | 5.3 |
| 163 | DMAEMA | 19.2 | 122 | BMA | 58 | PAA | 42 | 38 | 4.9 |
| 164 | DMAEMA | 36.6 | 233 | BMA | 54 | PAA | 46 | 44 | 5.7 |
| 165 | DMAEMA | 36.6 | 233 | BMA | 56 | PAA | 44 | 50 | 6.5 |
| 166 | DMAEMA | 49.6 | 315 | BMA | 50 | PAA | 50 | 59 | 7.6 |
| 167 | DMAEMA | 49.6 | 315 | BMA | 50 | PAA | 50 | 64 | 8.2 |
| 168 | DMAEMA | 49.6 | 315 | BMA | 46 | PAA | 54 | 56 | 7.1 |
| 169 | DMAEMA | 49.6 | 315 | BMA | 45 | PAA | 55 | 57 | 7.2 |
| 170 | DMAEMA | 45.5 | 289 | BMA | 53 | PAA | 47 | 56 | 7.2 |
| 171 | DMAEMA | 45.5 | 289 | BMA | 45 | PAA | 55 | 48 | 6.1 |
| 172 | DMAEMA | 45.5 | 289 | BMA | 52 | PAA | 48 | 54 | 6.9 |
| 173 | DMAEMA | 45.5 | 289 | BMA | 43 | PAA | 57 | 63 | 8.0 |
| 174 | DMAEMA | 19.2 | 122 | BMA | 55 | PAA | 45 | 40 | 5.2 |
| 175 | DMAEMA | 19.2 | 122 | BMA | 59 | PAA | 41 | 44 | 5.8 |
| 176 | DMAEMA | 36.6 | 233 | BMA | 54 | PAA | 46 | 52 | 6.7 |
| 177 | DMAEMA | 36.59 | 233 | BMA | 53 | PAA | 47 | 50 | 6.5 |
| 178 | DMAEMA | 49.57 | 315 | BMA | 43 | PAA | 57 | 49 | 6.2 |
| 179 | DMAEMA | 49.57 | 315 | BMA | 46 | PAA | 54 | 46 | 5.9 |
| 180 | DMAEMA | 49.57 | 315 | BMA | 44 | PAA | 56 | 42 | 5.3 |
| 181 | DMAEMA | 49.57 | 315 | BMA | 43 | PAA | 57 | 42 | 5.3 |
| 182 | DMAEMA | 45.46 | 289 | BMA | 50 | PAA | 50 | 46 | 5.9 |
| 183 | DMAEMA | 45.46 | 289 | BMA | 48 | PAA | 52 | 43 | 5.5 |
| 184 | DMAEMA | 45.46 | 289 | BMA | 45 | PAA | 55 | 43 | 5.4 |
| 185 | DMAEMA | 45.46 | 289 | BMA | 51 | PAA | 49 | 62 | 7.9 |
| 186 | DMAEMA | 19.161 | 122 | BMA | 59 | PAA | 41 | 38 | 5.0 |
| 187 | DMAEMA | 19.161 | 122 | BMA | 59 | PAA | 41 | 47 | 6.1 |
| 188 | DMAEMA | 36.59 | 233 | BMA | 54 | PAA | 46 | 45 | 5.8 |
| 189 | DMAEMA | 36.59 | 233 | BMA | 51 | PAA | 49 | 46 | 5.9 |
| 190 | DMAEMA | 49.57 | 315 | BMA | 43 | PAA | 57 | 39 | 4.9 |
| 191 | DMAEMA | 49.57 | 315 | BMA | 41 | PAA | 59 | 41 | 5.2 |
| 192 | DMAEMA | 49.57 | 315 | BMA | 44 | PAA | 56 | 47 | 6.0 |
| 193 | DMAEMA | 49.57 | 315 | BMA | 40 | PAA | 60 | 44 | 5.5 |
| 194 | DMAEMA | 45.46 | 289 | BMA | 48 | PAA | 52 | 40 | 5.1 |
| 195 | DMAEMA | 45.46 | 289 | BMA | 46 | PAA | 54 | 41 | 5.2 |
| 196 | DMAEMA | 45.46 | 289 | BMA | 47 | PAA | 53 | 41 | 5.2 |
| 197 | DMAEMA | 45.46 | 289 | BMA | 52 | PAA | 48 | 61 | 7.8 |
| 198 | DMAEMA | 19.161 | 122 | BMA | 62 | PAA | 38 | 43 | 5.6 |
| 199 | DMAEMA | 19.161 | 122 | BMA | 65 | PAA | 35 | 48 | 6.4 |
| 200 | DMAEMA | 36.59 | 233 | BMA | 50 | PAA | 50 | 41 | 5.3 |
| 201 | DMAEMA | 36.59 | 233 | BMA | 49 | PAA | 51 | 48 | 6.1 |
| 202 | DMAEMA | 49.57 | 315 | BMA | 41 | PAA | 59 | 44 | 5.5 |
| 203 | DMAEMA | 49.57 | 315 | BMA | 43 | PAA | 57 | 45 | 5.7 |
| 204 | DMAEMA | 49.57 | 315 | BMA | 42 | PAA | 58 | 44 | 5.6 |
| 205 | DMAEMA | 49.57 | 315 | BMA | 43 | PAA | 57 | 44 | 5.5 |
| 206 | DMAEMA | 45.46 | 289 | BMA | 42 | PAA | 58 | 43 | 5.5 |
| 207 | DMAEMA | 45.46 | 289 | BMA | 42 | PAA | 58 | 41 | 5.2 |
| 208 | DMAEMA | 45.46 | 289 | BMA | 46 | PAA | 54 | 40 | 5.1 |
| 209 | DMAEMA | 45.46 | 289 | BMA | 48 | PAA | 52 | 59 | 7.6 |
| 210 | DMAEMA | 19.161 | 122 | BMA | 66 | PAA | 34 | 48 | 6.3 |
| 211 | DMAEMA | 19.161 | 122 | BMA | 58 | PAA | 42 | 48 | 6.2 |
| 212 | DMAEMA | 36.59 | 233 | BMA | 50 | PAA | 50 | 46 | 6.0 |
| 213 | DMAEMA | 36.59 | 233 | BMA | 51 | PAA | 49 | 47 | 6.0 |
| 214 | DMAEMA | 49.57 | 315 | BMA | 38 | PAA | 62 | 45 | 5.6 |
| 215 | DMAEMA | 49.57 | 315 | BMA | 41 | PAA | 59 | 45 | 5.6 |
| 216 | DMAEMA | 49.57 | 315 | BMA | 38 | PAA | 62 | 46 | 5.8 |
| 217 | DMAEMA | 49.57 | 315 | BMA | 38 | PAA | 62 | 48 | 6.0 |
| 218 | DMAEMA | 45.46 | 289 | BMA | 44 | PAA | 56 | 48 | 6.0 |
| 219 | DMAEMA | 45.46 | 289 | BMA | 45 | PAA | 55 | 47 | 6.0 |
| 220 | DMAEMA | 45.46 | 289 | BMA | 47 | PAA | 53 | 44 | 5.5 |
| 221 | DMAEMA | 45.46 | 289 | BMA | 47 | PAA | 53 | 72 | 9.2 |

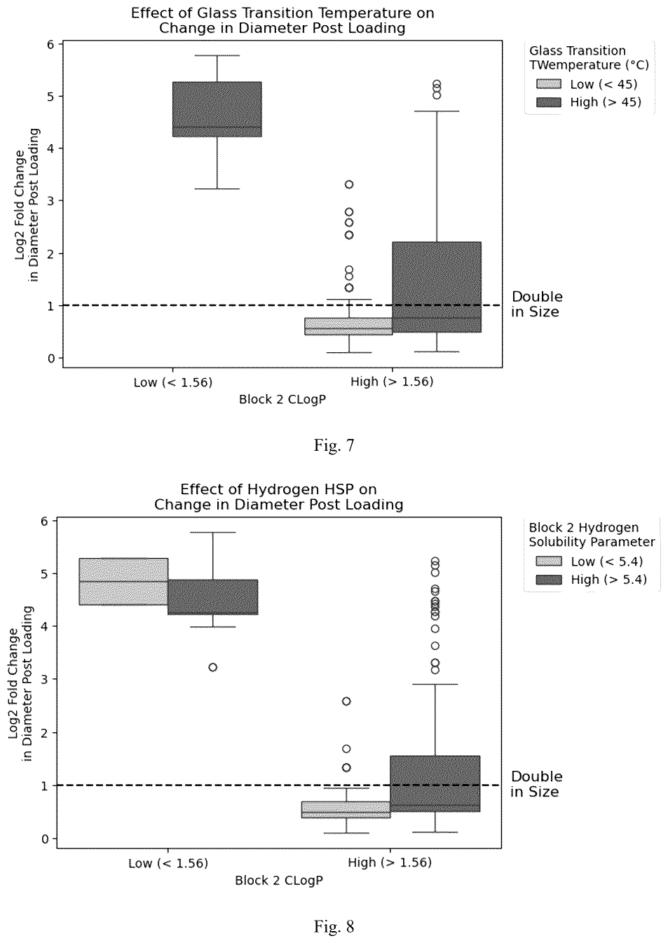

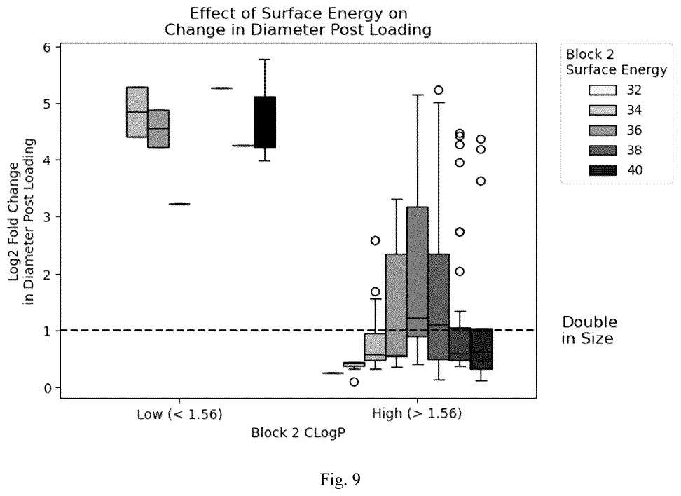

Table 3 shows the chemical descriptors for block 2 of the polymers. Estimated chemical descriptors including Hansen Solubility Parameters, glass transition temperature, surface energy, molar volume, density, oxygen permeability, refractive index, and dielectric constant were computed using the HSPiP software package. The C Log P of each monomer was assigned the value of X Log P3 listed in the PubChem database. For each block of the polymer, the values were calculated according to the measured monomer ratios shown in Table 2. Decision tree and gradient boosting tree algorithms were fit to the dataset using XGBoost and SciKit-Learn python packages. Feature importance from SHapley Additive exPlanations (SHAP) values were then used to identify the most important features when designing a polymer nanoparticle which less than doubles in size after loading. The results are shown in FIGS. 7-9, indicating that, in addition to C Log P of block 2, there were other factors that may impact the size of the loaded PNPs. For example, certain PNPs that retained their small size had a C Log P for block 2 of greater than 1.5. In addition, these PNPs also had Tg<45° C., Hydrogen HSP<5.4, and/or surface energy<34.

TABLE 3
Features of the PNPs,

Features

| | | B2 HSP | | |
|---|---|---|---|---|
| PNP ID | | Hydrogen | | B2 Surface |
| ID | CLogP B2 | Bonding | B2 Tg | Energy |
| 1 | 1.31 | 4.96 | 87.61 | 30.83 |
| 2 | 1.27 | 5.60 | 90.25 | 30.75 |
| 3 | 1.29 | 5.36 | 89.26 | 30.78 |
| 4 | 1.18 | 7.20 | 96.85 | 30.55 |
| 5 | 1.24 | 8.78 | 92.53 | 40.88 |
| 6 | 1.24 | 8.59 | 92.14 | 40.52 |
| 7 | 1.25 | 8.27 | 91.49 | 39.91 |
| 8 | 1.26 | 8.02 | 90.97 | 39.42 |
| 9 | 1.51 | 5.60 | 82.00 | 36.04 |
| 10 | 1.48 | 5.20 | 82.00 | 35.03 |
| 11 | 1.50 | 5.48 | 82.00 | 35.75 |
| 12 | 1.47 | 5.03 | 82.00 | 34.60 |
| 13 | 2.67 | 4.21 | 29.64 | 29.38 |
| 14 | 2.70 | 4.04 | 27.70 | 29.37 |
| 15 | 1.60 | 8.37 | 76.52 | 39.86 |
| 16 | 1.62 | 8.30 | 75.75 | 39.73 |
| 17 | 1.62 | 8.30 | 75.75 | 39.73 |
| 18 | 1.69 | 8.03 | 72.67 | 39.17 |
| 19 | 2.49 | 5.34 | 40.40 | 34.94 |
| 20 | 2.52 | 5.15 | 38.48 | 34.45 |
| 21 | 2.48 | 5.34 | 40.40 | 34.94 |
| 22 | 1.12 | 8.08 | 100.48 | 30.44 |
| 23 | 1.27 | 7.76 | 90.45 | 38.93 |
| 24 | 1.26 | 8.14 | 91.23 | 39.66 |
| 25 | 1.29 | 7.18 | 89.28 | 37.83 |
| 26 | 1.29 | 7.18 | 89.28 | 37.83 |
| 27 | 1.51 | 5.60 | 82.00 | 36.04 |
| 28 | 1.50 | 5.54 | 82.00 | 35.90 |
| 29 | 1.532 | 6.11 | 82.00 | 37.336 |
| 30 | 1.64 | 8.23 | 74.98 | 39.59 |
| 31 | 1.51 | 8.78 | 81.14 | 40.70 |
| 32 | 1.53 | 8.71 | 80.37 | 40.56 |
| 33 | 1.60 | 8.44 | 77.29 | 40.00 |
| 34 | 1.62 | 8.30 | 75.75 | 39.73 |
| 35 | 1.71 | 7.96 | 71.90 | 39.03 |
| 36 | 1.75 | 7.82 | 70.36 | 38.75 |
| 37 | 1.81 | 7.55 | 67.28 | 38.20 |
| 38 | 1.71 | 7.96 | 71.90 | 39.03 |
| 39 | 1.73 | 7.89 | 71.13 | 38.89 |
| 40 | 1.87 | 7.35 | 64.97 | 37.78 |
| 41 | 1.65 | 8.23 | 74.98 | 39.59 |
| 42 | 1.96 | 7.01 | 61.12 | 37.08 |
| 43 | 1.94 | 7.01 | 61.12 | 37.08 |
| 44 | 2.05 | 6.60 | 56.50 | 36.25 |
| 45 | 2.03 | 6.67 | 57.27 | 36.39 |
| 46 | 2.24 | 6.56 | 53.20 | 38.16 |
| 47 | 2.23 | 6.62 | 53.84 | 38.32 |
| 48 | 2.35 | 6.01 | 47.44 | 36.71 |
| 49 | 2.76 | 3.93 | 25.68 | 31.23 |
| 50 | 2.26 | 6.43 | 51.92 | 37.83 |
| 51 | 2.22 | 6.68 | 54.48 | 38.48 |
| 52 | 2.38 | 5.82 | 45.52 | 36.22 |
| 53 | 2.49 | 5.27 | 39.76 | 34.77 |
| 54 | 2.31 | 6.19 | 49.36 | 37.19 |
| 55 | 2.23 | 6.62 | 53.84 | 38.32 |
| 56 | 2.51 | 5.15 | 38.48 | 34.45 |
| 57 | 2.56 | 4.97 | 36.56 | 33.97 |
| 58 | 2.36 | 5.95 | 46.80 | 36.55 |
| 59 | 2.33 | 6.07 | 48.08 | 36.87 |
| 60 | 2.55 | 4.97 | 36.56 | 33.97 |
| 61 | 2.57 | 4.91 | 35.92 | 33.808 |
| 62 | 1.88 | 7.28 | 64.20 | 37.64 |
| 63 | 1.97 | 6.94 | 60.35 | 36.95 |
| 64 | 1.58 | 8.50 | 78.06 | 40.14 |
| 65 | 1.72 | 7.89 | 71.13 | 38.89 |
| 66 | 1.86 | 7.35 | 64.97 | 37.78 |
| 67 | 1.76 | 7.76 | 69.59 | 38.61 |
| 68 | 1.65 | 8.16 | 74.21 | 39.45 |
| 69 | 1.74 | 7.82 | 70.36 | 38.75 |
| 70 | 1.92 | 7.14 | 62.66 | 37.36 |
| 71 | 1.82 | 7.48 | 66.51 | 38.06 |
| 72 | 1.81 | 7.55 | 67.28 | 38.20 |
| 73 | 1.69 | 8.03 | 72.67 | 39.17 |
| 74 | 1.86 | 7.35 | 64.97 | 37.78 |
| 75 | 2.06 | 6.53 | 55.73 | 36.11 |
| 76 | 1.85 | 7.35 | 64.97 | 37.78 |
| 77 | 1.84 | 7.42 | 65.74 | 37.92 |
| 78 | 2.44 | 5.52 | 42.32 | 35.42 |
| 79 | 2.44 | 5.58 | 42.96 | 35.58 |
| 80 | 2.40 | 5.76 | 44.88 | 36.06 |
| 81 | 2.43 | 5.58 | 42.96 | 35.58 |
| 82 | 2.51 | 5.15 | 38.48 | 34.45 |
| 83 | 2.46 | 5.46 | 41.68 | 35.26 |
| 84 | 2.38 | 5.82 | 45.52 | 36.22 |
| 85 | 2.46 | 5.46 | 41.68 | 35.26 |
| 86 | 2.47 | 5.40 | 41.04 | 35.10 |
| 87 | 2.47 | 5.40 | 41.04 | 35.10 |
| 88 | 2.48 | 5.34 | 40.40 | 34.94 |
| 89 | 2.39 | 5.76 | 44.88 | 36.06 |
| 90 | 2.47 | 5.40 | 41.04 | 35.10 |
| 91 | 2.49 | 5.27 | 39.76 | 34.77 |
| 92 | 2.46 | 5.40 | 41.04 | 35.10 |
| 93 | 2.45 | 5.52 | 42.32 | 35.418 |
| 94 | 1.65 | 8.16 | 74.21 | 39.45 |
| 95 | 1.66 | 8.16 | 74.21 | 39.45 |
| 96 | 1.63 | 8.23 | 74.98 | 39.59 |
| 97 | 1.71 | 7.96 | 71.90 | 39.03 |
| 98 | 1.71 | 7.96 | 71.90 | 39.03 |
| 99 | 1.67 | 8.16 | 74.21 | 39.45 |
| 100 | 1.72 | 7.89 | 71.13 | 38.89 |
| 101 | 1.66 | 8.16 | 74.21 | 39.45 |
| 102 | 1.80 | 7.55 | 67.28 | 38.20 |
| 103 | 1.74 | 7.82 | 70.36 | 38.75 |
| 104 | 1.80 | 7.55 | 67.28 | 38.20 |
| 105 | 1.75 | 7.82 | 70.36 | 38.75 |
| 106 | 1.81 | 7.55 | 67.28 | 38.20 |
| 107 | 1.84 | 7.42 | 65.74 | 37.92 |
| 108 | 1.88 | 7.28 | 64.20 | 37.64 |
| 109 | 1.83 | 7.48 | 66.51 | 38.06 |
| 110 | 2.37 | 5.95 | 46.80 | 36.55 |
| 111 | 2.37 | 5.88 | 46.16 | 36.38 |
| 112 | 2.46 | 5.46 | 41.68 | 35.26 |
| 113 | 2.42 | 5.64 | 43.60 | 35.74 |
| 114 | 2.40 | 5.76 | 44.88 | 36.06 |
| 115 | 2.42 | 5.64 | 43.60 | 35.74 |
| 116 | 2.44 | 5.52 | 42.32 | 35.42 |
| 117 | 2.47 | 5.40 | 41.04 | 35.10 |
| 118 | 2.44 | 5.58 | 42.96 | 35.58 |
| 119 | 2.40 | 5.70 | 44.24 | 35.90 |
| 120 | 2.47 | 5.40 | 41.04 | 35.10 |
| 121 | 2.48 | 5.34 | 40.40 | 34.94 |
| 122 | 2.42 | 5.64 | 43.60 | 35.74 |
| 123 | 2.45 | 5.46 | 41.68 | 35.26 |
| 124 | 2.49 | 5.27 | 39.76 | 34.77 |
| 125 | 2.46 | 5.46 | 41.68 | 35.257 |
| 126 | 2.32 | 6.19 | 49.36 | 37.19 |
| 127 | 2.31 | 6.19 | 49.36 | 37.19 |
| 128 | 2.23 | 6.62 | 53.84 | 38.32 |
| 129 | 2.24 | 6.56 | 53.20 | 38.16 |
| 130 | 2.19 | 6.80 | 55.76 | 38.80 |
| 131 | 2.04 | 7.59 | 64.08 | 40.89 |
| 132 | 2.10 | 7.29 | 60.88 | 40.09 |
| 133 | 2.16 | 6.92 | 57.04 | 39.12 |
| 134 | 2.22 | 6.68 | 54.48 | 38.48 |
| 135 | 2.22 | 6.68 | 54.48 | 38.48 |
| 136 | 2.17 | 6.92 | 57.04 | 39.12 |
| 137 | 2.18 | 6.86 | 56.40 | 38.96 |
| 138 | 2.37 | 5.88 | 46.16 | 36.38 |
| 139 | 2.38 | 5.82 | 45.52 | 36.22 |
| 140 | 2.26 | 6.43 | 51.92 | 37.83 |
| 141 | 2.35 | 6.01 | 47.44 | 36.71 |
| 142 | 2.25 | 6.49 | 52.56 | 37.99 |
| 143 | 2.26 | 6.43 | 51.92 | 37.83 |
| 144 | 2.28 | 6.31 | 50.64 | 37.51 |
| 145 | 2.26 | 6.43 | 51.92 | 37.83 |
| 146 | 2.31 | 6.25 | 50.00 | 37.35 |
| 147 | 2.28 | 6.37 | 51.28 | 37.67 |
| 148 | 2.27 | 6.43 | 51.92 | 37.83 |
| 149 | 2.27 | 6.43 | 51.92 | 37.83 |
| 150 | 2.44 | 5.52 | 42.32 | 35.42 |
| 151 | 2.45 | 5.52 | 42.32 | 35.42 |
| 152 | 2.28 | 6.37 | 51.28 | 37.67 |
| 153 | 2.35 | 6.01 | 47.44 | 36.71 |
| 154 | 2.24 | 6.56 | 53.20 | 38.16 |
| 155 | 2.30 | 6.25 | 50.00 | 37.35 |
| 156 | 2.28 | 6.37 | 51.28 | 37.67 |
| 157 | 2.19 | 6.80 | 55.76 | 38.80 |
| 158 | 2.28 | 6.31 | 50.64 | 37.51 |
| 159 | 2.26 | 6.43 | 51.92 | 37.83 |
| 160 | 2.25 | 6.49 | 52.56 | 37.99 |
| 161 | 2.29 | 6.31 | 50.64 | 37.51 |
| 162 | 2.41 | 5.70 | 44.24 | 35.90 |
| 163 | 2.39 | 5.76 | 44.88 | 36.06 |
| 164 | 2.35 | 6.01 | 47.44 | 36.71 |
| 165 | 2.38 | 5.88 | 46.16 | 36.38 |
| 166 | 2.30 | 6.25 | 50.00 | 37.35 |
| 167 | 2.30 | 6.25 | 50.00 | 37.35 |
| 168 | 2.25 | 6.49 | 52.56 | 37.99 |
| 169 | 2.24 | 6.56 | 53.20 | 38.16 |
| 170 | 2.34 | 6.07 | 48.08 | 36.87 |
| 171 | 2.24 | 6.56 | 53.20 | 38.16 |
| 172 | 2.32 | 6.13 | 48.72 | 37.03 |
| 173 | 2.22 | 6.68 | 54.48 | 38.48 |
| 174 | 2.36 | 5.95 | 46.80 | 36.55 |
| 175 | 2.41 | 5.70 | 44.24 | 35.90 |
| 176 | 2.35 | 6.01 | 47.44 | 36.71 |
| 177 | 2.34 | 6.07 | 48.08 | 36.87 |
| 178 | 2.22 | 6.68 | 54.48 | 38.48 |
| 179 | 2.25 | 6.49 | 52.56 | 37.99 |
| 180 | 2.22 | 6.62 | 53.84 | 38.32 |
| 181 | 2.21 | 6.68 | 54.48 | 38.48 |
| 182 | 2.30 | 6.25 | 50.00 | 37.35 |
| 183 | 2.28 | 6.37 | 51.28 | 37.67 |
| 184 | 2.24 | 6.56 | 53.20 | 38.16 |
| 185 | 2.31 | 6.19 | 49.36 | 37.19 |
| 186 | 2.40 | 5.70 | 44.24 | 35.90 |
| 187 | 2.40 | 5.70 | 44.24 | 35.90 |
| 188 | 2.35 | 6.01 | 47.44 | 36.71 |
| 189 | 2.31 | 6.19 | 49.36 | 37.19 |
| 190 | 2.22 | 6.68 | 54.48 | 38.48 |
| 191 | 2.20 | 6.80 | 55.76 | 38.80 |
| 192 | 2.23 | 6.62 | 53.84 | 38.32 |
| 193 | 2.19 | 6.86 | 56.40 | 38.96 |
| 194 | 2.28 | 6.37 | 51.28 | 37.67 |
| 195 | 2.25 | 6.49 | 52.56 | 37.99 |
| 196 | 2.26 | 6.43 | 51.92 | 37.83 |
| 197 | 2.32 | 6.13 | 48.72 | 37.03 |
| 198 | 2.45 | 5.52 | 42.32 | 35.42 |
| 199 | 2.48 | 5.34 | 40.40 | 34.94 |
| 200 | 2.30 | 6.25 | 50.00 | 37.35 |
| 201 | 2.28 | 6.31 | 50.64 | 37.51 |
| 202 | 2.19 | 6.80 | 55.76 | 38.80 |
| 203 | 2.22 | 6.68 | 54.48 | 38.48 |
| 204 | 2.20 | 6.74 | 55.12 | 38.64 |
| 205 | 2.21 | 6.68 | 54.48 | 38.48 |
| 206 | 2.21 | 6.74 | 55.12 | 38.64 |
| 207 | 2.20 | 6.74 | 55.12 | 38.64 |
| 208 | 2.25 | 6.49 | 52.56 | 37.99 |
| 209 | 2.27 | 6.37 | 51.28 | 37.67 |
| 210 | 2.49 | 5.27 | 39.76 | 34.77 |
| 211 | 2.39 | 5.76 | 44.88 | 36.06 |
| 212 | 2.30 | 6.25 | 50.00 | 37.35 |
| 213 | 2.32 | 6.19 | 49.36 | 37.19 |
| 214 | 2.15 | 6.98 | 57.68 | 39.28 |
| 215 | 2.19 | 6.80 | 55.76 | 38.80 |
| 216 | 2.16 | 6.98 | 57.68 | 39.28 |
| 217 | 2.16 | 6.98 | 57.68 | 39.28 |
| 218 | 2.23 | 6.62 | 53.84 | 38.32 |
| 219 | 2.24 | 6.56 | 53.20 | 38.16 |
| 220 | 2.26 | 6.43 | 51.92 | 37.83 |
| 221 | 2.26 | 6.43 | 51.92 | 37.83 |

### Nucleic Acid Loading

In one example, mRNA was loaded into the PNPs by mixing the PNPs at a mass ratio of 30 to 1 (polymer to mRNA), and allowing the PNPs and mRNA to form an electrostatic complex at room temperature. The encapsulation efficiency was measured by performing a ribogreen assay, in which the ribogreen dye was added to the complexed sample and the fluorescence intensity measured. The encapsulation efficiency is calculated according to equation 1 below.

$$Encapsulation Efficiency ( \% ) = \frac{{Int}_{Free nucleic acid} - {Int}_{C o m p l e x}}{{Int}_{Free nucleic acid}}$$

Where Int_{Free nucleic acid} and Int_{Complex} are the fluorescence intensity of the ribogreen dye measured for the free nucleic acid, and the PNP complexed nucleic acid respectively (each at the same nucleic acid concentration). Tables 4 and 6 include replicates of the naked (nm) measurements. The particle size was measured for the loaded and unloaded PNPs using dynamic light scattering, shown in Table 4.

For analysis, the “Log Fold Difference” in PNP size was calculated upon loading with a nucleic acid as defined by the equation below.

$$Log Fold Difference = log ( \frac{{D}_{l o a d e d} - {D}_{u n l o a d e d}}{{D}_{u n l o a d e d}} )$$

The base 10 logarithm was used. The variables D_{unloaded} and D_{loaded} are the diameters of the PNPs, as measured by dynamic light scattering, of the particle without any payload, and after being complexed with the nucleic acid payload, respectively.

The “Log 2 Fold change” in PNP size upon loading with nucleic acid was calculated as defined by the equation below.

$$Log 2 Fold Change = {log}_{2} ( \frac{{D}_{l o a d e d}}{{D}_{u n l o a d e d}} )$$

The base 2 logarithm was used. As above, D_{unloaded} and D_{loaded} are the diameters of the PNPs, as measured by dynamic light scattering, of the particle without any payload, and after being complexed with the nucleic acid payload, respectively.

The Log Fold Difference, and the Log 2 Fold Change are calculated differently where the “Log Fold difference” is the Log base 10 of the difference in the size loaded and unloaded divided by the unloaded size. For this metric, a positive value indicates a doubling in size, while a negative value indicates a less than doubling in size upon loading. The “Log 2 Fold Change” is the log base 2 of the loaded size divided by the unloaded size. For this metric, a value greater than 1 indicates a doubling in size, while a value between 0 and 1 indicates a less than doubling in size. A negative value indicates a contraction in size upon loading.

TABLE 4
PNP diameter before and after mRNA loading

| | Calculated Output | | | |
|---|---|---|---|---|
| | | Size | log Fold | log 2 Fold |
| | Size | mRNA | Difference in | Change in |
| PNP ID | Unloaded | loaded | size after | size after |
| ID | (nm) | (nm) | mRNA loading | mRNA loading |
| 1 | 13.00 | 5544.00 | 2.63 | 8.74 |
| 2 | 7.00 | 1021.00 | 2.16 | 7.19 |
| 3 | 8.00 | 1098.00 | 2.13 | 7.10 |
| 4 | 7.00 | 1205.00 | 2.23 | 7.43 |
| 5 | 23.00 | 1267.00 | 1.73 | 5.78 |
| 6 | 17.00 | 1242.00 | 1.86 | 6.19 |
| 7 | 177.00 | 216.00 | −0.66 | 0.29 |
| 8 | 22.00 | 548.00 | 1.38 | 4.64 |
| 9 | 20.00 | 1078.00 | 1.72 | 5.75 |
| 10 | 21.00 | 824.00 | 1.58 | 5.29 |
| 11 | 25.00 | 734.00 | 1.45 | 4.88 |
| 12 | 28.00 | 596.00 | 1.31 | 4.41 |
| 13 | 12.00 | 1229.00 | 2.01 | 6.68 |
| 14 | 16.00 | 812.00 | 1.70 | 5.67 |
| 15 | 22.00 | 36.00 | −0.20 | 0.71 |
| 16 | 71.00 | 77.00 | −1.07 | 0.12 |
| 17 | 75.00 | 89.00 | −0.73 | 0.25 |
| 18 | 112.00 | 126.00 | −0.90 | 0.17 |
| 19 | 27.00 | 38.00 | −0.39 | 0.49 |
| 20 | 45.00 | 60.00 | −0.48 | 0.42 |
| 21 | 97.00 | 120.00 | −0.63 | 0.31 |
| 22 | 6.00 | 1781.00 | 2.47 | 8.21 |
| 23 | 18.00 | 693.00 | 1.57 | 5.27 |
| 24 | 29.00 | 554.00 | 1.26 | 4.26 |
| 25 | 57.00 | 351.00 | 0.71 | 2.62 |
| 26 | 212.00 | 359.00 | −0.16 | 0.76 |
| 27 | 35.00 | 655.00 | 1.25 | 4.23 |
| 28 | 82.00 | 225.00 | 0.24 | 1.46 |
| 29 | 49 | 457.00 | 0.92 | 3.22 |
| 30 | 28.08 | 584.17 | 1.30 | 4.38 |
| 31 | 26.58 | 423.21 | 1.17 | 3.99 |
| 32 | 26.80 | 594.45 | 1.33 | 4.47 |
| 33 | 21.74 | 270.53 | 1.06 | 3.64 |
| 34 | 27.16 | 495.80 | 1.24 | 4.19 |
| 35 | 23.51 | 454.01 | 1.26 | 4.27 |
| 36 | 21.65 | 481.49 | 1.33 | 4.48 |
| 37 | 37.23 | 44.09 | −0.74 | 0.24 |
| 38 | 22.78 | 492.30 | 1.31 | 4.43 |
| 39 | 35.74 | 551.11 | 1.16 | 3.95 |
| 40 | 43.17 | 52.87 | −0.65 | 0.29 |
| 41 | 23.41 | 30.54 | −0.52 | 0.38 |
| 42 | 29.75 | 574.35 | 1.26 | 4.27 |
| 43 | 50.22 | 455.79 | 0.91 | 3.18 |
| 44 | 148.16 | 214.56 | −0.35 | 0.53 |
| 45 | 66.49 | 95.88 | −0.35 | 0.53 |
| 46 | 15.81 | 597.51 | 1.57 | 5.24 |
| 47 | 19.58 | 514.34 | 1.40 | 4.72 |
| 48 | 16.98 | 601.16 | 1.54 | 5.15 |
| 49 | 70.60 | 84.18 | −0.72 | 0.25 |
| 50 | 16.39 | 527.80 | 1.49 | 5.01 |
| 51 | 21.09 | 465.88 | 1.32 | 4.47 |
| 52 | 18.28 | 42.83 | 0.13 | 1.23 |
| 53 | 33.80 | 42.92 | −0.57 | 0.34 |
| 54 | 21.95 | 555.68 | 1.39 | 4.66 |
| 55 | 109.34 | 170.79 | −0.25 | 0.64 |
| 56 | 48.98 | 61.31 | −0.60 | 0.32 |
| 57 | 46.86 | 60.84 | −0.53 | 0.38 |
| 58 | 57.95 | 107.94 | −0.06 | 0.90 |
| 59 | 28.05 | 40.41 | −0.36 | 0.53 |
| 60 | 147.42 | 158.53 | −1.12 | 0.10 |
| 61 | 69.79 | 111.92 | −0.22 | 0.68 |
| 62 | 39.31 | 54.75 | −0.41 | 0.48 |
| 63 | 54.86 | 113.68 | 0.03 | 1.05 |
| 64 | 27.42 | 41.99 | −0.27 | 0.62 |
| 65 | 28.47 | 58.80 | 0.03 | 1.05 |
| 66 | 236.84 | 480.99 | 0.01 | 1.02 |
| 67 | 62.96 | 85.47 | −0.45 | 0.44 |
| 68 | 30.45 | 45.50 | −0.31 | 0.58 |
| 69 | 45.21 | 62.70 | −0.41 | 0.47 |
| 70 | 173.23 | 403.73 | 0.12 | 1.22 |
| 71 | 197.11 | 293.09 | −0.31 | 0.57 |
| 72 | 78.66 | 95.65 | −0.67 | 0.28 |
| 73 | 98.44 | 138.33 | −0.39 | 0.49 |
| 74 | 131.51 | 145.21 | −0.98 | 0.14 |
| 75 | 114.33 | 754.04 | 0.75 | 2.72 |
| 76 | 55.52 | 113.16 | 0.02 | 1.03 |
| 77 | 117.39 | 276.61 | 0.13 | 1.24 |
| 78 | 32.07 | 46.11 | −0.36 | 0.52 |
| 79 | 40.13 | 203.83 | 0.61 | 2.34 |
| 80 | 31.58 | 45.84 | −0.35 | 0.54 |
| 81 | 28.13 | 41.19 | −0.33 | 0.55 |
| 82 | 34.69 | 46.98 | −0.45 | 0.44 |
| 83 | 38.18 | 83.06 | 0.07 | 1.12 |
| 84 | 38.25 | 55.30 | −0.35 | 0.53 |
| 85 | 35.30 | 51.89 | −0.33 | 0.56 |
| 86 | 67.36 | 85.63 | −0.57 | 0.35 |
| 87 | 102.58 | 259.24 | 0.18 | 1.34 |
| 88 | 44.90 | 67.27 | −0.30 | 0.58 |
| 89 | 48.67 | 69.10 | −0.38 | 0.51 |
| 90 | 128.40 | 247.56 | −0.03 | 0.95 |
| 91 | 161.15 | 518.44 | 0.35 | 1.69 |
| 92 | 52.11 | 68.59 | −0.50 | 0.40 |
| 93 | 50.56 | 67.57 | −0.47 | 0.42 |
| 94 | 36.53 | 60.86 | −0.18 | 0.74 |
| 95 | 32.25 | 41.61 | −0.54 | 0.37 |
| 96 | 47.06 | 96.20 | 0.02 | 1.03 |
| 97 | 38.06 | 50.32 | −0.49 | 0.40 |
| 98 | 67.68 | 131.60 | −0.02 | 0.96 |
| 99 | 48.98 | 69.32 | −0.38 | 0.50 |
| 100 | 83.12 | 143.34 | −0.14 | 0.79 |
| 101 | 61.54 | 93.55 | −0.28 | 0.60 |
| 102 | 115.92 | 703.87 | 0.71 | 2.60 |
| 103 | 97.79 | 405.41 | 0.50 | 2.05 |
| 104 | 185.38 | 760.06 | 0.49 | 2.04 |
| 105 | 82.67 | 208.61 | 0.18 | 1.34 |
| 106 | 168.91 | 854.29 | 0.61 | 2.34 |
| 107 | 182.32 | 1059.16 | 0.68 | 2.54 |
| 108 | 114.96 | 855.74 | 0.81 | 2.90 |
| 109 | 227.68 | 510.77 | 0.09 | 1.17 |
| 110 | 31.70 | 42.12 | −0.48 | 0.41 |
| 111 | 30.57 | 39.68 | −0.53 | 0.38 |
| 112 | 39.57 | 56.38 | −0.37 | 0.51 |
| 113 | 32.44 | 41.53 | −0.55 | 0.36 |
| 114 | 55.65 | 93.39 | −0.17 | 0.75 |
| 115 | 46.20 | 67.99 | −0.33 | 0.56 |
| 116 | 53.37 | 76.80 | −0.36 | 0.53 |
| 117 | 48.22 | 75.26 | −0.25 | 0.64 |
| 118 | 95.16 | 943.05 | 0.95 | 3.31 |
| 119 | 60.17 | 96.17 | −0.22 | 0.68 |
| 120 | 86.73 | 140.24 | −0.21 | 0.69 |
| 121 | 65.49 | 104.51 | −0.23 | 0.67 |
| 122 | 117.26 | 812.01 | 0.77 | 2.79 |
| 123 | 74.71 | 219.93 | 0.29 | 1.56 |
| 124 | 128.29 | 774.33 | 0.70 | 2.59 |
| 125 | 74.033 | 109.88 | −0.31 | 0.57 |
| 126 | 44.25 | 47.86 | −1.09 | 0.11 |
| 127 | N/A | N/A | N/A | N/A |
| 128 | 39.44 | 291.63 | 0.81 | 2.89 |
| 129 | N/A | N/A | N/A | N/A |
| 130 | 43.09 | 287.86 | 0.75 | 2.74 |
| 131 | N/A | N/A | N/A | N/A |
| 132 | N/A | N/A | N/A | N/A |
| 133 | N/A | N/A | N/A | N/A |
| 134 | 33.03 | 347.04 | 0.98 | 3.39 |
| 135 | N/A | N/A | N/A | N/A |
| 136 | N/A | N/A | N/A | N/A |
| 137 | N/A | N/A | N/A | N/A |
| 138 | 104.61 | 243.06 | 0.12 | 1.22 |
| 139 | N/A | N/A | N/A | N/A |
| 140 | 43.02 | 299.43 | 0.78 | 2.80 |
| 141 | N/A | N/A | N/A | N/A |
| 142 | 48.43 | 233.68 | 0.58 | 2.27 |
| 143 | N/A | N/A | N/A | N/A |
| 144 | 50.90 | 301.47 | 0.69 | 2.57 |
| 145 | N/A | N/A | N/A | N/A |
| 146 | N/A | N/A | N/A | N/A |
| 147 | N/A | N/A | N/A | N/A |
| 148 | N/A | N/A | N/A | N/A |
| 149 | N/A | N/A | N/A | N/A |
| 150 | N/A | N/A | N/A | N/A |
| 151 | N/A | N/A | N/A | N/A |
| 152 | N/A | N/A | N/A | N/A |
| 153 | N/A | N/A | N/A | N/A |
| 154 | N/A | N/A | N/A | N/A |
| 155 | N/A | N/A | N/A | N/A |
| 156 | N/A | N/A | N/A | N/A |
| 157 | N/A | N/A | N/A | N/A |
| 158 | N/A | N/A | N/A | N/A |
| 159 | N/A | N/A | N/A | N/A |
| 160 | N/A | N/A | N/A | N/A |
| 161 | N/A | N/A | N/A | N/A |
| 162 | N/A | N/A | N/A | N/A |
| 163 | N/A | N/A | N/A | N/A |
| 164 | N/A | N/A | N/A | N/A |
| 165 | N/A | N/A | N/A | N/A |
| 166 | N/A | N/A | N/A | N/A |
| 167 | N/A | N/A | N/A | N/A |
| 168 | N/A | N/A | N/A | N/A |
| 169 | N/A | N/A | N/A | N/A |
| 170 | N/A | N/A | N/A | N/A |
| 171 | N/A | N/A | N/A | N/A |
| 172 | N/A | N/A | N/A | N/A |
| 173 | N/A | N/A | N/A | N/A |
| 174 | 59.22 | 272.86 | 0.56 | 2.20 |
| 175 | N/A | N/A | N/A | N/A |
| 176 | N/A | N/A | N/A | N/A |
| 177 | N/A | N/A | N/A | N/A |
| 178 | 42.08 | 271.63 | 0.74 | 2.69 |
| 179 | N/A | N/A | N/A | N/A |
| 180 | N/A | N/A | N/A | N/A |
| 181 | N/A | N/A | N/A | N/A |
| 182 | N/A | N/A | N/A | N/A |
| 183 | N/A | N/A | N/A | N/A |
| 184 | N/A | N/A | N/A | N/A |
| 185 | N/A | N/A | N/A | N/A |
| 186 | N/A | N/A | N/A | N/A |
| 187 | N/A | N/A | N/A | N/A |
| 188 | N/A | N/A | N/A | N/A |
| 189 | N/A | N/A | N/A | N/A |
| 190 | N/A | N/A | N/A | N/A |
| 191 | N/A | N/A | N/A | N/A |
| 192 | N/A | N/A | N/A | N/A |
| 193 | N/A | N/A | N/A | N/A |
| 194 | N/A | N/A | N/A | N/A |
| 195 | N/A | N/A | N/A | N/A |
| 196 | N/A | N/A | N/A | N/A |
| 197 | N/A | N/A | N/A | N/A |
| 198 | N/A | N/A | N/A | N/A |
| 199 | N/A | N/A | N/A | N/A |
| 200 | N/A | N/A | N/A | N/A |
| 201 | N/A | N/A | N/A | N/A |
| 202 | N/A | N/A | N/A | N/A |
| 203 | N/A | N/A | N/A | N/A |
| 204 | N/A | N/A | N/A | N/A |
| 205 | N/A | N/A | N/A | N/A |
| 206 | N/A | N/A | N/A | N/A |
| 207 | N/A | N/A | N/A | N/A |
| 208 | N/A | N/A | N/A | N/A |
| 209 | N/A | N/A | N/A | N/A |
| 210 | 129.59 | 237.50 | −0.08 | 0.87 |
| 211 | N/A | N/A | N/A | N/A |
| 212 | 53.79 | 290.39 | 0.64 | 2.43 |
| 213 | N/A | N/A | N/A | N/A |
| 214 | 70.00 | 276.66 | 0.47 | 1.98 |
| 215 | N/A | N/A | N/A | N/A |
| 216 | N/A | N/A | N/A | N/A |
| 217 | N/A | N/A | N/A | N/A |
| 218 | 53.48 | 277.83 | 0.62 | 2.38 |
| 219 | N/A | N/A | N/A | N/A |
| 220 | N/A | N/A | N/A | N/A |
| 221 | N/A | N/A | N/A | N/A |

TABLE 5
PNP encapsulation percentage of mRNA
and transfection efficiency of mRNA

| | Encapsulation | Transfection |
|---|---|---|
| ID | Efficiency (%) | Efficiency (%) |
| 1 | 93 | 0.3 |
| 2 | 90 | 0.5 |
| 3 | 91 | 1.3 |
| 4 | 88 | 0.5 |
| 5 | 92 | 1.9 |
| 6 | 91 | 4.0 |
| 7 | 88 | 15.5 |
| 8 | 88 | 4.6 |
| 9 | 94 | 4.9 |
| 10 | 92 | 18.3 |
| 11 | 86 | 29.1 |
| 12 | 85 | 22.0 |
| 13 | 92 | 2.3 |
| 14 | 90 | 3.7 |
| 15 | 90 | 12.7 |
| 16 | 84 | 13.1 |
| 17 | 91 | 19.2 |
| 18 | 89 | 18.9 |
| 19 | 90 | 16.2 |
| 20 | 86 | 21.3 |
| 21 | 91 | 22.1 |
| 22 | 90 | 0.5 |
| 23 | 88 | 7.3 |
| 24 | 93 | 4.8 |
| 25 | 89 | 1.1 |
| 26 | 89 | 4.5 |
| 27 | 91 | 7.2 |
| 28 | 92 | 6.1 |
| 29 | 90 | 4.7 |

TABLE 6
PNP diameter before and after DNA loading

| | Calculated Output | | | |
|---|---|---|---|---|
| | | Size | log Fold | log 2 Fold |
| | Size | pDNA | Difference in | Change in |
| PNP ID | Unloaded | loaded | size after | size after |
| ID | (nm) | (nm) | pDNA loading | pDNA loading |
| 1 | 13.00 | 2812.00 | 2.33 | 7.76 |
| 2 | 7.00 | 2413.00 | 2.54 | 8.43 |
| 3 | 8.00 | 2640.00 | 2.52 | 8.37 |
| 4 | 7.00 | 2764.00 | 2.60 | 8.63 |
| 5 | 23.00 | 727.00 | 1.49 | 4.98 |
| 6 | 17.00 | 263.00 | 1.16 | 3.95 |
| 7 | 177.00 | 229.00 | −0.53 | 0.37 |
| 8 | 22.00 | 232.00 | 0.98 | 3.40 |
| 9 | 20.00 | 337.00 | 1.20 | 4.07 |
| 10 | 21.00 | 272.00 | 1.08 | 3.70 |
| 11 | 25.00 | 239.00 | 0.93 | 3.26 |
| 12 | 28.00 | 272.00 | 0.94 | 3.28 |
| 13 | 12.00 | 2869.00 | 2.38 | 7.90 |
| 14 | 16.00 | 412.00 | 1.39 | 4.69 |
| 15 | 22.00 | 278.00 | 1.07 | 3.66 |
| 16 | 71.00 | 207.00 | 0.28 | 1.54 |
| 17 | 75.00 | 185.00 | 0.17 | 1.30 |
| 18 | 112.00 | 189.00 | −0.16 | 0.75 |
| 19 | 27.00 | 284.00 | 0.98 | 3.39 |
| 20 | 45.00 | 319.00 | 0.78 | 2.83 |
| 21 | 97.00 | 177.00 | −0.08 | 0.87 |
| 22 | 6.00 | 2897.00 | 2.68 | 8.92 |
| 23 | 18.00 | 454.00 | 1.38 | 4.66 |
| 24 | 29.00 | 251.00 | 0.88 | 3.11 |
| 25 | 57.00 | 266.00 | 0.56 | 2.22 |
| 26 | 212.00 | 323.00 | −0.28 | 0.61 |
| 27 | 35.00 | 305.00 | 0.89 | 3.12 |
| 28 | 82.00 | 293.00 | 0.41 | 1.84 |
| 29 | 49 | 239.00 | 0.59 | 2.29 |

### Dynamic Light Scattering

The particle size of the polymers, with and without nucleic acid payload complexed, was measured via dynamic light scattering using a Wyatt DynaPro III.

### NMR Calculation of Molecular Weight

For each polymer synthesized, the NMR spectra was measured to calculate the relative amounts of each monomeric building block in the final diblock copolymer, as adapted from the methods described in J. Chem. Educ., 2011, 88, 1098-1104. Using the molecular weight of the first block, as measured by gel permeation chromatography, these molar ratios measured from NMR, along with the molecular weights of the monomeric repeat units, were used to calculate the molecular weight of the diblock copolymer, which is reported in Table 2 as NMR measured Mw in kilodaltons.

### Calculation of the C Log P of a Polymer Block

For each polymer synthesized, the theoretical C Log P was calculated of a given block by calculating the molar weighted average of the X Log P3 of the monomers used in that block.

Calculation of the Log Relative Fold Difference in PNP Size Upon Loading with Nucleic Acid

For analysis, the “Log Fold Difference” in PNP size was calculated upon loading with a nucleic acid as defined by the equation below.

$$Log Fold Difference = log ( \frac{{D}_{l o a d e d} - {D}_{u n l o a d e d}}{{D}_{u n l o a d e d}} )$$

Where D_{unloaded} and D_{loaded} are the diameters of the PNPs, as measured by dynamic light scattering, of the particle without any payload, and after being complexed with the nucleic acid payload, respectively.

### Transfection Efficiency in HEK293 Cells

To measure the in vitro transfection efficiency, the PNPs were loaded with an mRNA expressing Cre recombinase protein. These were dosed into HEK293 cells that have been modified to express a loxP cassette. Untreated, the cells express green fluorescent protein (GFP). However, if the mRNA is successfully delivered and expressed, the GFP expression is knocked out, and red fluorescent protein (RFP) is expressed. Cells were seeded in 96 well plates at 40,000 cells per well and incubated for 48 hours before treating with 150 ng of mRNA per well, loaded into PNPs at a mass ratio of 20 to 1 (polymer to mRNA). The number of RFP and GFP expressing cells were counted via flow cytometry, and the transfection efficiency was calculated as defined by the equation below.

$$Transfection efficiency ( \% ) = \frac{RFP expressing Cells}{Total Cells}$$

1. A block copolymer comprising
a first block having a monomer unit represented by formula I: 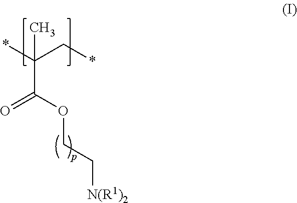
or a salt thereof, wherein:
p is 0 or an integer selected from 1-3,
each R¹ is individually selected from H or alkyl; and
wherein each * represents a connecting point to the rest of the block copolymer; and
a copolymer second block comprising a copolymer of monomer units selected from formula II: 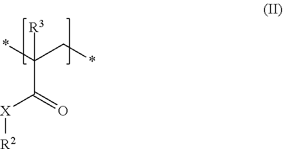
or a salt thereof, wherein:
X is —O—;
R² is alkyl; and
R³ is alkyl;
wherein each * represents a connecting point to the rest of the block copolymer; and
formula III: 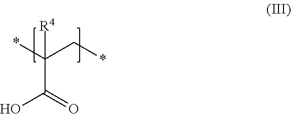
or a salt thereof, wherein:
R⁴ is alkyl;
wherein each * represents a connecting point to the rest of the block copolymer.

2. The block copolymer of claim 1, wherein p is 1.

3. The block copolymer of claim 1 or 2, wherein each R¹ is C₁-C₃ alkyl.

4. The block copolymer of claim 1 or 2, wherein each R¹ is methyl.

5. The block copolymer of any one of claims 1-4, wherein R² is C₁-C₆ alkyl.

6. The block copolymer of any one of claims 1-5, wherein R² is butyl.

7. The block copolymer of any one of claims 1-6, wherein R³ is C₁-C₆ alkyl.

8. The block copolymer of any one of claims 1-7, wherein R³ is methyl.

9. The block copolymer of any one of claims 1-8, wherein R⁴ is C₁-C₆ alkyl.

10. The block copolymer of any one of claims 1-9, wherein R⁴ is ethyl or propyl.

11. The block copolymer of any one of claims 1-10, wherein R⁴ is ethyl.

12. The block copolymer of any one of claims 1-10, wherein R⁴ is propyl.

13. A block copolymer comprising a first block and a second block, wherein:
the first block comprises a homopolymer of poly dimethylaminoethyl methacrylate p(DMAEMA), and
the second block comprises a copolymer of an alkylacrylate (e.g., butylmethacrylate (BMA)) and an acrylic acid (e.g., ethylacrylic acid (EAA) or propylacrylic acid (PAA));
wherein the first block is about 40% to about 90% by weight of the block copolymer.

14. The block copolymer of any one of the preceding claims, wherein the first block has a molecular weight of about 4,000 Da to about 50,000 Da, for example about 4,000 Da to about 40,000, about 4,000 Da to about 30,000, or about 4,000 Da to about 20,000.

15. The block copolymer of any one of the preceding claims, wherein the first block has a molecular weight of about 4,000 Da to about 13,000 Da.

16. The block copolymer of any one of the preceding claims, wherein the first block has a molecular weight of about 5,000 Da to about 10,000 Da.

17. The block copolymer of any one of the preceding claims, wherein the copolymer has a total molecular weight of about 5 kDa to about 25 kDa.

18. The block copolymer of any one of the preceding claims, wherein the copolymer has a total molecular weight of about 5 kDa to about 20 kDa.

19. The block copolymer of any one of the preceding claims, wherein the first block has a degree of polymerization of about 30 to about 150.

20. The block copolymer of any one of the preceding claims, wherein the second block has a degree of polymerization of about 15 to about 60.

21. The block copolymer of any one of the preceding claims, wherein the second block has a degree of polymerization of about 15 to about 25.

22. The block copolymer of any one of claims 1-20, wherein the second block has a degree of polymerization of about 25 to about 35.

23. The block copolymer of any one of claims 1-20, wherein the second block has a degree of polymerization of about 35 to about 45.

24. The block copolymer of any one of claims 1-20, wherein the second block has a degree of polymerization of about 45 to about 55.

25. The block copolymer of any one of the preceding claims, wherein the second block has a molecular weight of about 1 kDa to about 13 kDa.

26. The block copolymer of any one of claims 1-24, wherein the second block has a molecular weight of about 2 kDa to about 5 kDa.

27. The block copolymer of any one of claims 1-24, wherein the second block has a molecular weight of about 2 kDa to about 4 kDa.

28. The block copolymer of any one of claims 1-24, wherein the second block has a molecular weight of about 3 kDa to about 5 kDa.

29. The block copolymer of any one of the preceding claims, wherein the second block has:
(i) a C Log P greater than about 1.5,
(ii) a Tg less than about 45° C.,
(iii) a hydrogen HSP less than about 5.4,
(iv) a surface energy of less than about 34,
(v) a surface energy of greater than about 38, or
(vi) a combination thereof with the exception of a combination of (iv) and (v).

30. The block copolymer of any one of the preceding claims, wherein the second block has a C Log P greater than about 1.5.

31. The block copolymer of any one of the preceding claims, wherein the second block has a C Log P of at least 1.6.

32. The block copolymer of any one of the preceding claims, wherein the second block has a C Log P of at least 1.7.

33. The block copolymer of any one of claims 1-29, wherein the second block has a C Log P of about 1.5 to about 3.

34. The block copolymer of any one of claims 1-29, wherein the second block has a C Log P of about 1.5 to about 2.6.

35. The block copolymer of claims 1-29, wherein the second block has a C Log P of about 1.6 to about 2.6.

36. The block copolymer of any one of claims 29-35, wherein the second block has at least two of (i)-(v), for example a C Log P greater than about 1.5 and a Tg less than about 45° C., or a C Log P greater than about 1.5 and a hydrogen HSP less than about 5.4.

37. The block copolymer of any one of claims 29-35, wherein the second block has at least three of (i)-(v), for example a C Log P greater than about 1.5, a Tg less than about 45° C., and a hydrogen HSP less than about 5.4, or a C Log P greater than about 1.5, a hydrogen HSP less than about 5.4 and a surface energy of less than about 34.

38. The block copolymer of any one of the preceding claims, wherein the molar ratio of the monomer units of the compound of formula II and the compound of formula III in the second block is about 30:70 to about 90:10.

39. The block copolymer of any one of the preceding claims wherein the molar ratio of the monomer units of the compound of formula II and the compound of formula III in the second block is about 50:50 to about 75:25.

40. The block copolymer of any one of the preceding claims, wherein the molar ratio of the monomer units of the compound of formula II and the compound of formula III in the second block is about 60:40.

41. The block copolymer of any one of the preceding claims, wherein the first block comprises a cap of formula: 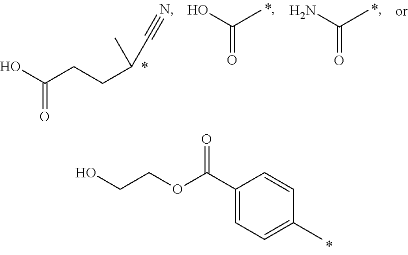
or a salt thereof, wherein * represents a point of covalent attachment to the first block.

42. The block copolymer of any one of the preceding claims, wherein the first block comprises a cap of formula: 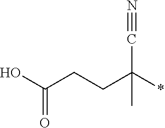 [SMILES: *C(C)(C#N)CCC(=O)O]
or a salt thereof, wherein the * represents a point of connection to the first block.

43. The block copolymer of any one of the preceding claims, wherein the second block comprises a cap of formula: 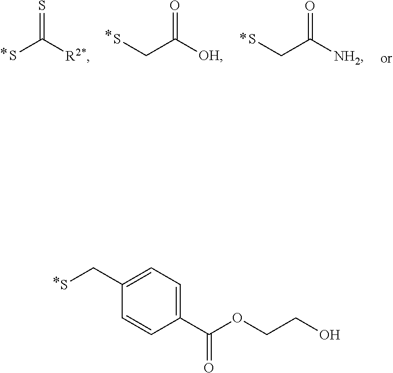
or a salt thereof, wherein * represents a point of covalent attachment to the second block, and R²* is —SC₂-C₁₂ alkyl or C₆H.

44. The block copolymer of any one of the preceding claims, wherein the second block comprises a cap of formula: 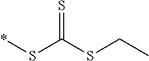 [SMILES: *SC(=S)SCC]
or a salt thereof, wherein the * represents a point of connection to the second block.

45. A polymer nanoparticle comprising:
the block copolymer according to any one of claims 1-44,
wherein the nanoparticle has a diameter of about 15 nm to about 250 nm.

46. The polymer nanoparticle of claim 45, wherein the nanoparticle has a diameter of about 20 nm to about 115 nm.

47. The polymer nanoparticle of claim 45, wherein the nanoparticle has a diameter of about 20 nm to about 100 nm.

48. The polymer nanoparticle of claim 45, wherein the nanoparticle has a diameter of about 20 nm to about 50 nm.

49. The polymer nanoparticle of claim 45, wherein the nanoparticle has a diameter of about 50 nm to about 115 nm.

50. A composition comprising:
a polymer nanoparticle according to any one of claims 45-49, and
a nucleic acid complexed to the polymer nanoparticle.

51. The composition of claim 50, wherein the polymer nanoparticle is complexed to the nucleic acid via electrostatic interaction.

52. The composition according to claim 50 or 51, wherein the composition has a diameter, and the diameter of the composition is less than about two-fold greater than the diameter of the nanoparticle without the nucleic acid.

53. The composition according to claim 50 or 51, wherein the composition has a diameter, and the diameter of the composition is less than about 1.5-fold greater than the diameter of the nanoparticle without the nucleic acid.

54. The composition according to claim 50 or 51, wherein the composition has a diameter, and the diameter of the composition is less than about 1.2-fold greater than the diameter of the nanoparticle without the nucleic acid.

55. The composition according to claim 50 or 51, wherein the composition has a diameter, and the diameter of the composition is about 1 fold to about 2 fold greater than the diameter of the nanoparticle without the nucleic acid.

56. The composition according to claim 50 or 51, wherein the composition has a diameter, and the diameter of the composition is about 1 fold to about 1.5 fold greater than the diameter of the nanoparticle without the nucleic acid.

57. The composition according to claim 50 or 51, wherein the composition has a diameter, and the diameter of the composition is about 1 fold to about 1.2 fold greater than the diameter of the nanoparticle without the nucleic acid.

58. The composition of any one of claims 50-57, wherein the nucleic acid is an RNA (e.g., mRNA).

59. The composition of any one of claims 50-57, wherein the nucleic acid is a DNA (e.g., pDNA).

60. The composition of any one of claims 50-59, wherein the percent encapsulation is at least about 80%.

61. The composition of any one of claims 50-59, wherein the percent encapsulation is at least about 80% to about 95%.

62. The composition of any one of claims 50-61, wherein the ratio of nanoparticle to nucleic acid is about 5:1 to about 60:1.

63. The composition of any one of claims 50-61, wherein the ratio of nanoparticle to nucleic acid is about 10:1 to about 60:1.

64. The composition of any one of claims 50-61, wherein the ratio of nanoparticle to nucleic acid is about 10:1 to about 30:1.

65. The composition of any one of claims 50-64, further comprising a pharmaceutically acceptable carrier.

66. A method of treating a disease (e.g., a neurological disease) in a patient in need thereof, the method comprising:
administering a therapeutically effective amount of a composition according to any one of claims 50-65.

67. A method of transfecting a cell, the method comprising:
contacting the cell with an effective amount of a composition according to any one of claims 50-65.

68. The method of claim 67, wherein the method is in vitro.

69. The method of claim 67, wherein the method is in vivo.
